(19) United States
(12) Patent Application Publication
(10) Pub. No.: US 2008/0181971 A1
(43) Pub. Date: Jul. 31, 2008

(54) Combination Cancer Treatments Comprising Elsamitrucin and Other Agents

(76) Inventors: Guru Reddy, Irvine, CA (US); Luigi Lenaz, Newton, PA (US)

Correspondence Address: KIRKPATRICK & LOCKHART PRESTON GATES ELLIS LLP, 1900 MAIN STREET, SUITE 600, IRVINE, CA, 92614-7319, US

(21) Appl. No.: 12/020,338

(22) Filed: Jan. 25, 2008

Related U.S. Application Data
(60) Provisional application No. 60/886,603, filed on Jan. 25, 2007.

(51) Int. Cl. A61K 33/24; A61K 31/70; A61P 35/00; A61K 31/704

(52) U.S. Cl. 424/649; 514/25; 514/27

(57) ABSTRACT

Disclosed herein are methods relating to combination cancer treatments that include elsamitrucin and other agents.

### CROSS REFERENCE TO RELATED APPLICATIONS

This application is a continuation of U.S. Patent Application No. 60/886,603 filed on Jan. 25, 2007, the entire disclosure of which is incorporated herein by reference.

### FIELD OF THE INVENTION

The present invention relates to combination cancer treatments that comprise elsamitrucin and other agents.

### BACKGROUND OF THE INVENTION

Despite the numerous advances in cancer treatment, there is still no universally effective and acceptable treatment. One problem that remains with presently available treatments is that the doses required to produce a therapeutic effect also produce unacceptable side effects. One avenue to improve currently available treatments, then, is to attempt to combine lower doses of more than one cancer treatment in an attempt to identify a combination that can produce therapeutic effects with fewer or less severe side effects. The present invention, therefore, examines a variety of combinations of potential cancer treatments. More specifically, the present invention examines the effectiveness of a variety of potential cancer treatments in combination with elsamitrucin.

### SUMMARY OF THE INVENTION

Eight studied drug combinations produced from additive to moderately synergistic activities in at least one tested cell line. These results illustrate that the outcome of combination therapies may be more favorable than the use of single-agent therapies.

Specifically, one embodiment according to the present invention includes a method of treating cancer comprising administering elsamitrucin with one or more of 5-fluorouracil, bortezomib, camptothecin, carmustine, cisplatin, doxorubicin, etoposide, gemcitabine, methotrexate, and paclitaxel. In another embodiment, elsamitrucin is administered with paclitaxel. In another embodiment, elsamitrucin is administered with cisplatin. In another embodiment according to the present invention, the elsamitrucin comprises a salt form. In another embodiment, the salt form is a tosylate salt form or a succinate salt form.

Methods according to the present invention can be used to treat mammals. In one embodiment, the mammal is selected from the group consisting of a human, a dog, a cat, a hamster, a guinea pig, a ferret and a pig.

### BRIEF DESCRIPTION OF THE TABLES

Table 1 shows the growth inhibitory activities of elsamitrucin and selected anticancer agents against the HT29 human colon carcinoma cell line.

Table 2 and Table 2 (Supplement) show the values for elsamitrucin and selected anticancer agents, individually and in combination, for growth inhibition of the HT29 human colon carcinoma cell line.

Table 3 and Table 3 (Supplement) show the combination index values for elsamitrucin and selected anticancer agents with the HT29 human colon carcinoma cell line.

Table 4 shows the growth inhibitory activities of elsamitrucin and selected anticancer agents against the SKMES human non-small cell lung carcinoma cell line.

Table 5 and Table 5 (Supplement) show the Dₘ values for elsamitrucin and second agents, individually and in combination, for growth inhibition of SKMES human non-small cell lung carcinoma cells.

Table 6 and Table 6 (Supplement) show combination index values for elsamitrucin and selected anticancer agents with the SKMES human non-small cell lung carcinoma cell line.

Table 7 shows growth inhibitory activities of elsamitrucin and selected anticancer agents against the Daudi human lymphoma cell line.

Table 8 and Table 8 (Supplement) show the Dₘ values for elsamitrucin and second agents, individually and in combination, for growth inhibition of Daudi human lymphoma cells.

Table 9 and Table 9 (Supplement) show combination index values for elsamitrucin and selected anticancer agents with the Daudi human lymphoma cell line.

Table 10 shows the protocol design for the described HCT116-e256 study.

Table 11 shows the treatment response summary for the described HCT116-e256 study.

### BRIEF DESCRIPTION OF THE FIGURES

FIGS. 1A and 1B show the antiproliferative activities of elsamitrucin, cisplatin and their combination against human HT29 colon carcinoma cells in culture (SPA-13 HT29-e26 Plate 1).

FIGS. 2A and 2B show the antiproliferative activities of elsamitrucin, bortezomib and their combination against human HT29 colon carcinoma cells in culture (SPA-16 HT29-e27 Plate 3).

FIGS. 3A and 3B show the antiproliferative activities of elsamitrucin, etoposide and their combination against human HT29 colon carcinoma cells in culture (SPA-07 HT29-e23 Plate 3).

FIGS. 4A and 4B show isobol plots for mutually exclusive (left panel) and mutually non-exclusive (right panel) drug-target interactions in cultured human HT29 colon carcinoma cells for elsamitrucin in combination with cisplatin (4A; SPA-13 HT29-e26 Plate 1) and bortezomib (4B; SPA-16 HT29-e27 Plate 3).

FIG. 5 shows the individual times to endpoint for male mice in the described HCT116-e256 study.

FIGS. 6A and 6B show the median tumor growth and Kaplan-Meier plot for the elsamitrucin monotherapy groups in the described HCT116-e256 study.

FIGS. 7A and 7B show the median tumor growth and Kaplan-Meier plot for the elsamitrucin and paclitaxel groups in the described HCT116-e256 study.

FIG. 8 shows the median tumor growth plot for the elsamitrucin and paclitaxel groups in the described HCT116-e256 study with Group 8 not truncated.

FIGS. 9A and 9B show the median tumor growth and Kaplan-Meier plot for the elsamitrucin and cisplatin groups in the described HCT116-e256 study.

### DETAILED DESCRIPTION OF THE INVENTION

As previously stated, currently available cancer therapies or treatments are not universally effective or acceptable due to the production of adverse side effects at many doses that are required to achieve a therapeutic effect. Therefore, one avenue to improve cancer treatments could be to find a combination of cancer treatments that can be used in combination at lower doses. If an acceptable combination was found, the elements of the combination could be individually given in smaller amounts and work together to produce a therapeutic effect while not producing as many or as severe of side effects as presently seen with their individual administration. Alternatively, even if given at the same amounts as generally given individually, the combination of various cancer agents could produce synergistic therapeutic effects. The present invention, then, examined a variety of potential cancer treatment combinations. More specifically, the present invention examined the effectiveness of a variety of anti-cancer agents in combination with elsamitrucin.

Elsamitrucin is a heterocyclic antineoplastic antibiotic isolated from the gram positive bacterium Actinomycete strain J907-21 as described in U.S. Pat. Nos. 4,518,589 and 4,572,895 which are incorporated herein by reference for all they disclose related to the natural history, chemical composition, methods of preparing and bioactivity of elsamitrucin. Elsamitrucin intercalates into DNA at guanine-cytosine (G-C)-rich sequences and inhibits topoisomerase I and II, resulting in single-strand breaks and inhibition of DNA replication. Elsamitrucin possesses significant oncolytic activity against metastatic cancer of the breast, colon and rectum, non-small cell lung and ovary and in patients with relapsed or refractory non-Hodgkin's lymphoma.

Elsamitrucin is known chemically as benzo(h)(1)benzopyrano(5,4,3-cde)(1)ebnzopyran-5,12-dione,10((2-O-(2-amino-2,6-dideoxy-3-O-methyl-alpha-D-galactopyranosyl)-6-deoxy-3-C-methyl-beta-D-galactopyranosyl)oxy)-6-hydroxy-1-methyl. Elsamitrucin is also known as 10-O-elsaminosylelsarosylchartarin, BBM 2478A, BMY-28090, SPI-28090, BRN 5214813, elsamicin A, elsamitrucina, and elsamitrucine.

The following experiments described in Example 1 were conducted to determine whether combinations of elsamitrucin with other anticancer drugs produce additive, synergistic, or antagonistic growth inhibitory activity against cultured human tumor cells.

### EXAMPLE 1

### In Vitro Growth Inhibitory Activities of Ten Anticancer Drugs, Individually and in Combinations with Elsamitrucin, against Human HT29 Colon Carcinoma, SKMES Non-Small Cell Lung Carcinoma and Daudi Lymphoma Cells

Ten chemotherapeutic drugs were selected for combination with elsamitrucin: 5-fluorouracil, bortezomib, camptothecin, carmustine, cisplatin, doxorubicin, etoposide, gemcitabine, methotrexate, and paclitaxel.

5-fluorouracil is a fluorinated pyrimidine that inhibits the normal production of thymidine. Bortezomib is a 26S proteasome inhibitor that prevents the degradation of intracellular proteins, affecting signaling cascades. Camptothecin is a topoisomerase I inhibitor. Carmustine, nitrosourea, alkylates and cross-links DNA and inhibits DNA repair. Cisplatin is a DNA cross-linker. Doxorubicin generates free radicals and inhibits DNA topoisomerase II. Etoposide initiates single strand DNA breaks, inhibits topoisomerase II and binds to DNA. Gemcitabine inhibits DNA synthesis and apoptosis. Methotrexate inhibits the function of DNA, RNA and overall protein synthesis. Paclitaxel promotes the formation of microtubules, preventing depolymerization and inhibiting cellular replication.

These individual agents and their combinations were evaluated for in vitro activity against the HT29 colon carcinoma, SKMES non-small cell lung carcinoma, and Daudi B-lymphoblastic cell lines. Cell growth in 96-well microculture plates was determined with a semi-automated MTT assay. Efficacies of drug combinations were analyzed according to the methods of Chou and Talalay, utilizing CalcuSyn software. Twenty-one in vitro experiments were conducted to determine the individual and combined growth inhibitory activities of elsamitrucin and the ten selected agents in the three cell lines.

### Materials and Methods

a. Chemicals, Culture Media, and Supplements. Elsamitrucin was provided by Spectrum Pharmaceuticals (Irvine, Calif.). 5-fluorouracil (Fluorouracil Injection) and cisplatin (CISplatin Injection, sodium chloride) were purchased from American Pharmaceutical Partners, Inc. (Schaumberg, Ill.); bortezomib (Velcade for Injection, mannitol boronic ester) from Millenium (Cambridge, Mass.); camptothecin from SIGMA (St. Louis, Mo.); carmustine (BiCNU) from Bristol Laboratories (New York, N.Y.); doxorubicin from Meiji Seika Kaisha, Ltd. (Tokyo, Japan); etoposide (Etoposide Injection 20 mg/mL in 65% PEG300, 30.5% benzyl alcohol, 8% Polysorbate 80, and 0.2% citric acid) from Bedford Laboratories (Bedford, Ohio); gemcitabine (Gemzaro, Eli Lilly & Co., 38 mg/mL) from Eli Lilly & Co. (Indianapolis, Ind.); methotrexate from Xanodyne Pharmacal, Inc. (Newport, Ky.); and paclitaxel from Mayne Group Ltd. (Melbourne, AU). Elsamitrucin, camptothecin, carmustin, and paclitaxel were dissolved in dimethyl sulfoxide (DMSO), and diluted with the cell culture medium to prepare stock solutions containing 1% DMSO. 5-Fluorouracil, bortezomib, cisplatin, doxorubicin, etoposide, gemcitabine, and methotrexate were dissolved in or diluted with medium containing 1% DMSO. MTT (3-[4,5-dimethylthiazol-2-yl]2,5-diphenyltetrazolium bromide) was obtained from Sigma Chemical Co. RPMI-1640 medium, antibiotic antimycotic 100× (consisting of 10,000 units/mL penicillin G sodium, 10,000 pg/mL streptomycin sulfate, and 25 pg/mL amphotericin B (fungizone)), glutamine (200 mM), HEPES buffer (1 M), gentamicin (50 mg/mL), sodium bicarbonate (7.5%), sodium pyruvate (100 mM), and fetal bovine serum were obtained from Gibco BRL (Gaithersburg, Md.). The complement in fetal bovine serum was inactivated by heating at 56° C. for 30 min.

b. Cell Lines. The human HT29 colon carcinoma, SKMES non-small cell lung carcinoma, and Daudi B-lymphoblastic cell lines were originally obtained from ATCC (American Type Culture Collection). These cell lines have been maintained and stored as frozen stocks. The tumor cells were cultured in RPMI-1640 medium supplemented with 10% fetal bovine serum, 100 units/mL penicillin G sodium, 100 pg/mL streptomycin sulfate, 0.25 pg/mL amphotericin B, 25 pg/mL gentamicin, 2 mM glutamine, 10 mM HEPES, and 0.075% sodium bicarbonate. The medium for Daudi cells was additionally supplemented with 1 mM sodium pyruvate. The doubling times for the HT29, SKMES, and Daudi cell lines were approximately 33, 27, and 38 hr, respectively.

c. Growth Inhibition Assay. Anti-proliferative activities against the HT29, SKMES, and Daudi cell lines were evaluated in vitro by the MTT assay. Cells (1,500-2,000 cells/well) were seeded in a 96-well microculture plate in 180 pL of medium/well. After overnight incubation of the plates in a humidified chamber at 37° C. with 5% CO₂ and 95% air, test agent solutions were added to each well in 20 pL volumes. DMSO was present in all wells at a final concentration of 0.1%. The cells were incubated with the test agents in a humidified chamber at 37° C., with 5% CO₂ and 95% air. When the appropriate cell density was attained in the control wells, the plates were centrifuged briefly, and 100 pL of the growth medium was removed. The cell cultures were incubated with 50 μL of MTT reagent (1 mg/mL in Dulbecco's phosphate-buffered saline) for 4 hr at 37° C. The resultant purple formazan precipitate was solubilized with 200 μL of 0.04 N HCl in isopropanol.

Absorbance was monitored at a wavelength of 595 nm, and at a reference wavelength of 650 nm, using a Tecan GENios microplate reader. The results of each experiment were stored in Excel format.

d. Preliminary Experiments: Design and Data Analysis. The concentration causing 50% growth inhibition (IC₅₀) was determined for each agent against each of the cell lines. The initial experiments utilized serial three-fold dilutions of the drugs to produce ten test concentrations over a broad concentration range. Based on the initial results, a narrower concentration range was selected, and serial 1:1.5 dilutions were utilized to produce ten test concentrations. Experiments were repeated as needed.

Absorbance readings at each drug concentration were converted to percent of control and the results were imported into Prism 3.03 (GraphPad) for nonlinear regression analysis. IC₅₀ values were estimated with Prism 3.03 by fitting the data to the sigmoidal dose-response curve described by the following four-logistic equation:

$$Y = \frac{Top - Bottom}{1 + {( \frac{X}{{IC}_{50}} )}^{n}} + Bottom$$

where Top is the maximal percent of control absorbance, Bottom is the minimal percent of control absorbance at the highest agent concentration, Y is the percent of control absorbance, X is the agent concentration, IC₅₀ is the concentration of agent that inhibits cell growth by 50% compared to the control cells, and n is the slope of the curve.

For assays that utilized lower dilution factors to produce narrower concentration ranges, the results were analyzed with both Prism and CalcuSyn (as described below) to allow comparison of the IC₅₀ and Da, values. The results guided the selection of appropriate concentration ranges and concentration ratios for the combination experiments.

e. Combination Experiments: Design and Data Analysis. The fixed-ratio design of Chou was used for the combination experiments. For each experiment, the drug solutions were mixed at a ratio that was expected to provide approximately equipotent concentrations, and the mixture was serially diluted with medium containing 1% DMSO to yield 10× stock solutions of the test concentrations. The differences between the highest and lowest drug concentrations were 11.4 fold, 5.6 fold, and 3.8 fold for serial dilutions of 1:1.5, 1:1.33, and 1:1.25, respectively. Each agent was tested individually and in combination at seven concentrations. The concentration range selected for each assay was designed to bracket the concentrations that produced half-maximal effects (ED₅₀) with each single agent and their mixture. The concentrations used for the single-drug assays were twice as high as their concentrations in the mixture. Each assay was performed in quadruplicate. For each drug combination, the individual agents and their mixture were assayed on the same 96-well plate. The effects of the individual drugs and their combinations were analyzed by the method of Chou and Talalay. The results of each experiment were entered into CalcuSyn (BIOSOFT) for dose-effect analysis. The fraction of affected drug targets, Fa, at a given drug concentration, was determined from the fractional decrease in absorbance at 595 nm by the following equation:

$${F}_{a} = \frac{{A}_{control} - {A}_{drug}}{{A}_{control}} \qquad ( 1 )$$

where A_{control} and A_{drug} are the respective absorbances of vehicle control and drug-treated wells in the MIT assay.

The 50% growth inhibitory concentration (IC₅₀), which is termed the median-effect dose, Dₘ, was obtained from the antilog of the X-intercept of a plot of X=log(D) versus Y=log [Fa/(I−Fa)] according to the logarithmic form of the median-effect equation:

log [Fₐ/(1−Fₐ)]=m log(D)−m log(Dₘ) (2)

where the slope, m, is related to the sigmoidicity of the dose-effect curve, with m=1, >1, and <1 indicating hyperbolic, sigmoidal, and reverse sigmoidal shapes, respectively. For each Fa obtained with a mixture, the concentrations of individual drugs producing the same Fa are computed from the following form of the median-effect equation:

D=Dₘ[Fₐ/(1−Fₐ)]^{1/m} (3)

From the ratios of the concentrations of individual and combined drugs yielding the same Fa, a combination index, CI, was computed for mutually exclusive inhibitors from:

$$CI = \frac{{( D )}_{1}}{{( {D}_{x} )}_{1}} + \frac{{( D )}_{2}}{{( {D}_{x} )}_{2}} \qquad ( 4 )$$

and for mutually non-exclusive inhibitors from:

$$CI = \frac{{( D )}_{1}}{{( {D}_{x} )}_{1}} + \frac{{( D )}_{2}}{{( {D}_{x} )}_{2}} + \frac{{( D )}_{1} {( D )}_{2}}{{( {D}_{x} )}_{1} {( {D}_{x} )}_{2}} \qquad ( 5 )$$

where (D)₁ and (D)₂ are the actual concentrations of drug 1 and drug 2 in a mixture inhibiting growth by x %, and (Dx)₁ and (Dx)₂ are the calculated concentrations of the individual drugs causing the same x % inhibition.

For complex interactions between two drugs and their target(s), indicated by non-parallel regression lines on the median-effect plot, CalcuSyn computes CI values for both exclusive and non-exclusive drug interactions. Using Chou and Talalay definitions, CI value ranges indicate varying degrees of antagonism, which are listed in the following table.

| CI Values | Type and Degree of Interaction |
|---|---|
| <0.1 | very strong synergism |
| 0.1-0.3 | strong synergism |
| 0.3-0.7 | synergism |
| 0.7-0.85 | moderate synergism |
| 0.85-0.90 | slight synergism |
| 0.90-1.10 | nearly additive |
| 1.10-1.20 | slight antagonism |
| 1.20-1.45 | moderate antagonism |
| 1.45-3.3 | antagonism |
| 3.3-10 | strong antagonism |
| >10 | very strong antagonism |

Although “antagonism” is associated with CI values of 1.45-3.3 in this table, the term may also be used to indicate any less-than-additive interaction, without specification of strength.

### Results

As stated, the HT29 colon carcinoma, SKMES lung carcinoma, and Daudi lymphoma cell lines were utilized to characterize the individual and combined antiproliferative activities of elsamitrucin and ten selected anticancer agents. Ten preliminary single-agent experiments determined the potency of each agent in each cell line. Eleven combination experiments determined whether combination of elsamitrucin with 5-fluorouracil, bortezomib, camptothecin, carmustine, cisplatin, doxorubicin, etoposide, gemcitabine, methotrexate, or paclitaxel produced additive, synergistic, or antagonistic antiproliferative effects. For each cell line, the results of all single-agent assays and ten representative combination assays are reported in Tables 1-9 and discussed below. The results of all plate combination assays, successful and unsuccessful, are presented in Supplemental Tables 2, 3, 5, 6, 8, and 9.

a. Growth Inhibitory Activities of Elsamitrucin, Alone and in Combination with Ten Anticancer Agents, against Human HT29 Colon Carcinoma Cells (SPA-01, SPA-04, SPA-07, SPA-10, SPA-12, SPA-13, SPA-16, and SPA-20)

To provide initial estimates of IC₅₀ values for growth inhibition of HT29 colon carcinoma cells, elsamitrucin and ten selected anticancer drugs were tested over broad concentration ranges (SPA-01, Table 1). Based on these estimates, a narrower concentration range was selected for the second experiment with each agent (SPA-04, Table 1). In cases where the minimum or maximum drug concentration was not appropriate, the concentration range was readjusted and a third experiment was performed (SPA-10, Table 1). The data from the second and third experiments were analyzed with both Prism and CalcuSyn software. The IC₅₀ values determined from nonlinear regression analyses (Prism) of the concentration response curves (% of control absorbance versus log concentration) are similar to the Dₘ values determined by linear regression analysis of the median-effect plots (CalcuSyn).

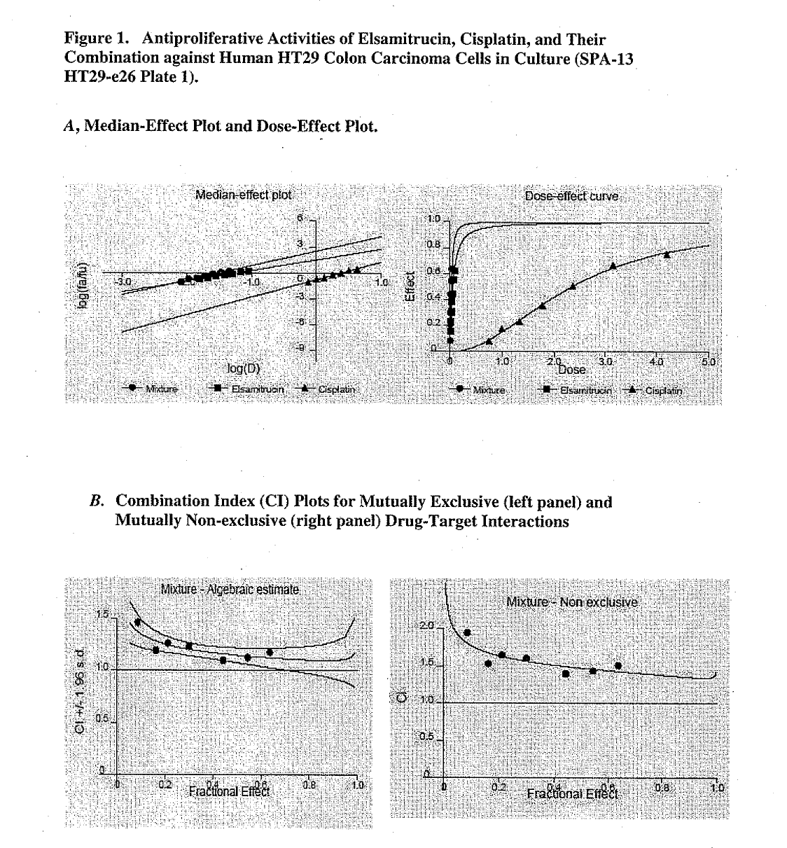

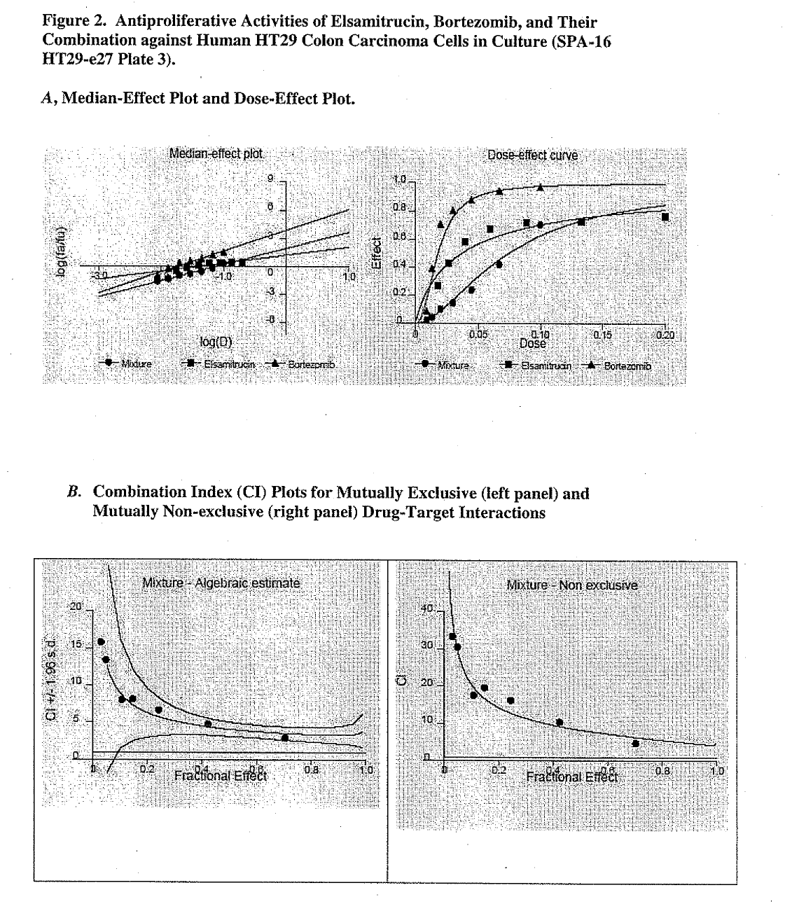

Elsamitrucin was mixed with each second agent at a ratio expected to provide equipotent concentrations. Twenty-two plate assays were analyzed to evaluate the individual drugs and their combinations in HT29 cells. The Dₘ values from ten selected assays, one for each drug combination, are presented in Table 2. The table also lists the drug concentration ratios, the serial dilutions that yielded the seven test concentrations, and the concentration ranges of the individual drugs in each assay. Results from two representative assays are presented in FIGS. 1 and 2. FIG. 1A shows the median-effect and dose-effect plots for elsamitrucin and cisplatin as single agents and when combined at a concentration ratio of 1:46.7. FIG. 2A shows the corresponding plots for elsamitrucin and bortezomib, individually and in a 1:0.5 combination.

Based on the results shown in Table 2, elsamitrucin inhibited HT29 cell proliferation with an average Dₘ of 0.063 pM (range=0.036-0.118 pM). This average value is quite similar to the average Dₘ of 0.077 pM determined in the preliminary experiments (Table 1). The calculated Dₘ values (pM) for the second drugs as single agents were: 5-fluorouracil, 0.79; bortezomib, 0.018; camptothecin, 0.02; carmustine, 130; cisplatin, 2.3; doxorubicin, 0.048; etoposide, 0.71; gemcitabine, 0.0040; methotrexate, 0.022; and paclitaxel, 0.0032.

With eight drug combinations, the Dₘ values for one or both agents were higher for the single agents than for the mixtures. With two combinations, however, Dₘ increased for one or both agents: the values for both agents in the elsamitrucin/bortezomib combination increased approximately two fold, and the value for 5-fluorouracil increased only slightly.

Plots of experimental CI values from analyses based on mutually exclusive and mutually non-exclusive drug-target interactions for the elsamitrucin/cisplatin combination are shown in FIG. 1B. The corresponding plots for the elsamitrucin/bortezomib combination are shown in FIG. 2B. Calculated CI values were obtained from the algebraic average of the experimentally determined CI values. On each plot for mutually exclusive interactions, the middle curve represents the simulated data from which the calculated CI values for these combinations were derived. The upper and lower curves in the same plots show 95% confidence levels. A single curve appears on the plots for mutually non-exclusive interactions because CalcuSyn does not statistically evaluate those results.

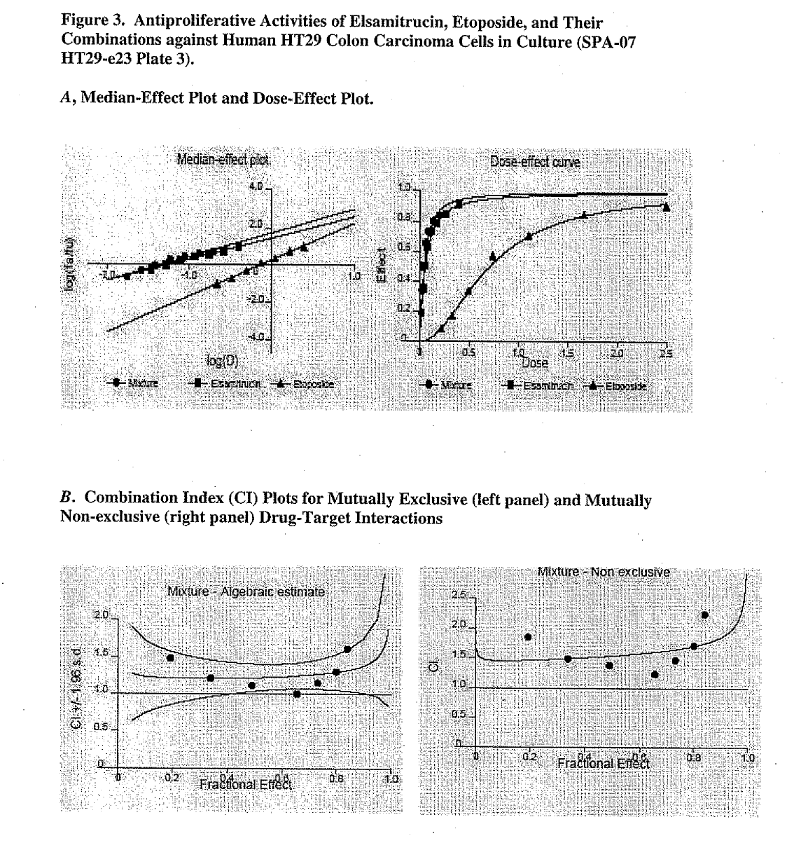

In FIGS. 1B and 2B, the curves for the simulated data provide good fits for the experimental CI values obtained with the cisplatin and bortezomib combinations. In some cases, however, the data simulation curve indicates the average antagonism over the entire effect range, but does not provide a good fit for the experimental data. FIG. 3 presents CalcuSyn plots for the elsamitrucin/etoposide combination, which illustrate such a case. In FIG. 3A, the experimental data for the drug mixture deviate systematically from a straight line on the median-effect plot. This indicates that the median-effect equation does not adequately describe the relationship between drug concentration and response. In these studies, nonlinear median-effect data tend to produce experimental CI values that lie on a curve, as shown in FIG. 3A, and the experimental CI values tend to increase at both ends of the concentration range (FIG. 3B). The experimentally determined CI values fall on a smooth concave-upward curve whose shape is not replicated by the curve for the simulated data.

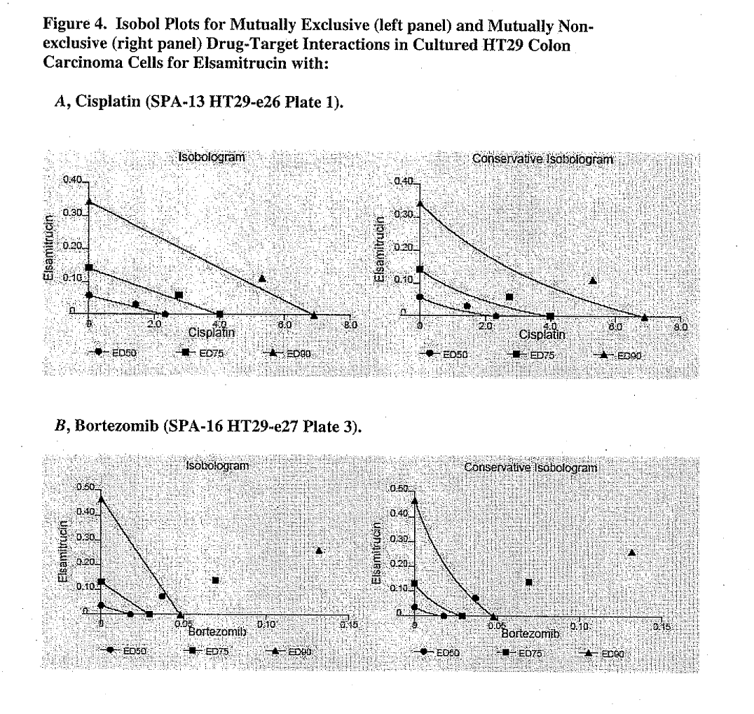

Table 3 presents the calculated combination index (CI) values at Fa=0.5, 0.75, and 0.9 (ED₅₀, ED₇₅, and ED₉₀) for the mutually exclusive and mutually non-exclusive interactions of the ten drug combinations in HT29 cells. According to analyses of mutually exclusive activities, some degree of antagonism, i.e., CI>1.1, was observed with every drug combination. The interactions varied with the effect level, and their descriptions are listed as ranging from ED₅₀ to ED₉₀. The interactions range from overall slight antagonism (CI=1.15-1.10) with cisplatin (see FIG. 1B), to overall strong antagonism (CI=4.1-3.32) with bortezomib (see FIG. 2B). The results for the cisplatin and bortezomib combinations are illustrated in the isobol plots in FIGS. 4A and 4B. In these plots, straight lines connect the single-drug concentrations that produced 50%, 75%, and 90% growth inhibition. Points falling on the straight lines indicate all combinations of drug concentrations that might produce the same percent inhibition via additive effects. Points that fall above these lines indicate antagonism; points below the lines indicate synergism.

Only two combinations yielded additive effects with elsamitrucin: interactions with 5-fluorouracil ranged widely from antagonistic to nearly additive (CI=1.56-0.99); interactions with camptothecin ranged from moderately antagonistic to nearly additive (CI=1.25-0.90), based on analyses of mutually exclusive interactions.

The calculated CI values for mutually non-exclusive interactions are consistently somewhat higher than those for mutually exclusive interactions. Additive activity is indicated only for camptothecin at the ED₉₀ level (Table 3).

b. Growth Inhibitory Activities of Elsamitrucin, Alone and in Combination with Ten Anticancer Agents, against Human SKMES Non-Small Cell Carcinoma Cells (SPA-02, SPA-03, SPA-06, SPA-09, SPA-11, SPA-14, and SPA-17)

The antiproliferative activities of elsamitrucin, the selected anticancer drugs, and their combinations against human SKMES non-small cell lung cancer cells were characterized as described above for the HT29 cell line. The IC₅₀ and Dₘ values determined from four preliminary experiments with elsamitrucin and the selected agents in the SKMES cell line are listed in Table 4 (SPA-02, SPA-03, SPA-09, and SPA-14). The Dₘ values determined in ten optimal assays selected from three combination experiments (SPA-06, SPA-11, and SPA-17) are summarized in Table 5. In these ten assays, elsamitrucin inhibited proliferation with an average Dₘ of 0.022 pM (range=0.011-0.053 μM). The calculated Dₘ values (pM) for the second drugs as single agents were: 5-fluorouracil, 4.4; bortezomib, 0.016; camptothecin, 0.008; carmustine, 70; cisplatin, 1.7; doxorubicin, 0.056; etoposide, 0.24; gemcitabine, 0.0028; methotrexate, 0.0082; and paclitaxel, 0.0035. The Dₘ values for all agents were approximately the same or lower when they were tested in the mixtures than when they were assayed as single agents.

The calculated CI values for mutually exclusive and mutually non-exclusive drug-target interactions in SKMES cells are listed in Table 6. Based on the calculated values for mutually exclusive interactions, combination of elsamitrucin with each of these agents at ED₅₀ produced results ranging from slight antagonism with cisplatin to antagonism with 5-fluorouracil, camptothecin, doxorubicin, etoposide, and methotrexate. With five agents, there is a shift towards additivity or synergy at higher effect levels. The calculated CI values for the bortezomib combination indicate additive activity at ED₇₅, and moderate synergism at ED₉₀. At ED₉₀, the doxorubicin combination produced slight synergism, and combinations with carmustine, cisplatin, and gemcitabine produced additive effects. Mutually non-exclusive analyses always yielded higher CI values; nevertheless, slight synergism with bortezomib, and additivity with doxorubicin, were observed at ED₉₀.

C. Growth Inhibitory Activities of Elsamitrucin, Alone and in Combination with Ten Anticancer Agents, Against Human Daudi Lymphoma Cells (SPA-05, SPA-08, SPA-15, SPA-18, SPA-19, and SPA-21)

The effects of elsamitrucin, ten selected anticancer drugs, and their combinations on the proliferation of human Daudi B-lymphoblasts were characterized as described above for HT29 cells. The IC₅₀ and Dₘ values determined in three preliminary experiments with the Daudi cell line (SPA-05, SPA-08, and SPA-15) are listed in Table 7. The Dₘ values determined in ten optimal assays selected from three combination experiments (SPA-18, SPA-19, and SPA-21) are summarized in Table 8. In these ten assays, elsamitrucin inhibited proliferation with an average Dₘ of 0.027 μM (range=0.013-0.034 μM). The approximate Dₘ values (μM) for the second drugs as single agents were: 5-fluorouracil, 11.7; bortezomib, 0.0039; camptothecin, 0.0093; carmustine, 42; cisplatin, 0.49; doxorubicin, 0.009; etoposide, 0.047; gemcitabine, 0.0012; methotrexate, 0.045; and paclitaxel, 0.004. Dₘ values were almost always lower in the combinations, and none increased (Table 8).

The calculated CI values for mutually exclusive and mutually non-exclusive interactions between the drug pairs are listed in Table 9. Based on the calculated CI values for mutually exclusive interactions at ED₅₀, ED₇₅, and ED₉₀, combination of elsamitrucin with etoposide produced overall additive activity. Additive interactions occurred with three other combinations: with cisplatin and doxorubicin at ED₅₀ and ED₇₅; and with camptothecin at ED₉₀. All other combinations produced some degree of antagonism at one or more effect levels. The most unfavorable interactions occurred with methotrexate, which was antagonistic at all effect levels.

### Discussion

The studies described in Example 1 investigated the interactions between the growth inhibitory activities of elsamitrucin and each of ten selected anticancer drugs in cultured human HT29 colon carcinoma, SKMES non-small cell lung carcinoma, and Daudi lymphoma cells. Based on calculated CI values at ED₅₀, ED₇₅, and ED₉₀ for inhibition of cell proliferation, concurrent exposure of these cells to the ten drug combinations yielded predominantly less-than-additive effects. Eight drug combinations produced additive, slightly synergistic, and/or moderately synergistic activities at one or more effect levels in at least one cell line; however, these interactions were not observed in all three cell lines. The elsamitrucin/cisplatin combination produced the most consistently favorable interactions, although results differed somewhat with the three cell lines. In HT29 cells, there was slight antagonism across all effect levels (Table 3, FIGS. 1 and 4A)). In SKMES cells, the cisplatin interaction ranged from slightly antagonistic to nearly additive (Table 6). In Daudi cells, it ranged from additive to moderately antagonistic (Table 9). The Dₘ for cisplatin decreased by 38% in HT29 cells, 47% in SKMES cells, and 59% in Daudi cells (Tables 2, 5, and 8). Concomitantly, the Dₘ for elsamitrucin in this combination also decreased in the three cell lines. It may be of interest to determine whether the relatively favorable interactions between elsamitrucin and cisplatin can be reproduced in other human tumor cell lines, and whether carboplatin and oxaliplatin yield similar outcomes.

With most drugs, potency varied with the target tumor cell line. Despite inter-experimental variations in the Dₘ values, it is evident that HT29 cells were less sensitive to growth inhibition than the other cell lines. The average Dₘ for elsamitrucin was 0.063 μM for HT29 cells, compared to 0.022 and 0.027 μM for SKMES and Daudi cells, respectively (Tables 2, 5, and 8). The single-agent Dₘ values for five other drugs (camptothecin, carmustine, cisplatin, etoposide, and gemcitabine) were highest in the HT29 cells; however, HT29 was the most sensitive cell line for growth inhibition by 5-fluorouracil.

Overall, drug interactions tended to be more antagonistic in HT29 cells than in SKMES and Daudi cells, yielding generally higher CI values (Tables 3, 6, and 9). Also, while bortezomib produced moderate antagonism to moderate synergism in SKMES cell cultures with mutually exclusive analysis, strong antagonism was observed at all effect levels in HT29 cell cultures (FIGS. 2 and 4B). With HT29 cells, the Dₘ values for elsamitrucin and bortezomib increased approximately two-fold, and the Dₘ for 5-fluorouracil increased slightly in the drug mixtures, while the values for one or both drugs in seven combinations were lower. In SKMES cells, the Dₘ values for one or both drugs decreased for all ten drug combinations, while a moderate increase (less than 1.5 fold) was observed with camptothecin. In Daudi cells, both Dₘ values were lower for all drug combinations.

Although moderate antagonism or antagonism was the most frequent outcome of mutually exclusive analyses, the Dₘ values for elsamitrucin and/or the second drug decreased in most assays. Thus, at concentrations equal to half their single-agent Dₘ values, these drug combinations produced more than 50% inhibition. These results illustrate that, even for moderately antagonistic combinations, the outcome of combination therapy may be more favorable than that of either single-agent therapy, albeit less so than if the interaction were additive or synergistic.

Analyses based on mutually non-exclusive interactions yielded results that are skewed in the direction of greater antagonism, relative to analyses based on exclusive interactions. Given the complexity of whole-cell systems, neither totally exclusive nor totally non-exclusive inhibition would be expected to occur. In addition, different drug interactions might occur with some combinations if different drug ratios were employed. Since drug effects were determined at approximately equipotent concentrations, the present study provides only a snapshot of all potential drug interactions. Finally, these ten combination treatments were concurrent, and different interactions might be observed if tumor cells were exposed to sequential drug treatments.

### EXAMPLE 2

### Evaluation of Elsamitrucin in Combination with Cisplatin and Paclitaxel in the HCT116 Human Colon Carcinoma in Nude Mice

Next, the effect of elsamitrucin against the HCT116 human colon carcinoma as a monotherapy and in combination with paclitaxel or cisplatin was evaluated.

### Materials and Methods

The study described in Example 2 employed eleven groups of mice including an untreated control group, monotherapy groups receiving 10 or 5 mg/kg elsamitrucin, 15 or 7.5 mg/kg paclitaxel, or 2.7 or 1.35 mg/kg cisplatin, and combination therapy groups administered elsamitrucin with paclitaxel or cisplatin combined at the lower or higher of the monotherapy dose levels. The dose levels chosen for paclitaxel and cisplatin were subtherapeutic in monotherapy. Tumors were measured twice weekly during the experiment, and each animal was euthanized when its tumor reached the endpoint volume of 2000 mm³ or on the last day of the study (Day 59), whichever came first.

Treatment outcome was assessed by tumor growth delay (TGD), defined as the difference in median time to endpoint tumor burden in a treatment group compared to a control group.

### Methods

a. Mice. Female nude mice (nu/nu, Harlan) were 8 to 9 weeks old and had body weights ranging from 15.1 to 26.6 g on Day 1 of the study. The animals were fed ad libitum water (reverse osmosis, 1 ppm CI) and NIH 31 Modified and Irradiated Lab Diet® consisting of 18.0% crude protein, 5.0% crude fat, and 5.0% crude fiber. The mice were housed on irradiated ALPHA-Dri® Bed-O'Cobs® Laboratory Animal Bedding in static microisolators on a 12-hour light cycle at 21-22° C. (70-72° F.) and 40-60% humidity. Testing facilities complied with the recommendations of the Guide for Care and Use of Laboratory Animals with respect to restraint, husbandry, surgical procedures, feed and fluid regulation, and veterinary care. The animal care and use program at the testing facility is accredited by AAALAC, which assures compliance with accepted standards for the used and care of laboratory animals.

b. Tumor Implantation. Tumors were initiated from HCT116 tumor cells cultured in RPMI-1640 medium supplemented with 10% heat inactivated fetal bovine serum, 100 units/mL penicillin G, 100 μg/mL streptomycin sulfate, 0.25 μg/mL amphotericin B, 2 mM glutamine, and 25 μg/mL gentamicin. Tumor cells were maintained in tissue culture flasks in a humidified incubator at 37° C. in an atmosphere of 5% CO₂ and 95% air. On the day of tumor implant, the cells were trypsinized, harvested by centrifugation and resuspended in phosphate buffered saline. Each mouse received 5×10⁶ HCT116 tumor cells in 0.2 mL implanted subcutaneously in the right flank, and tumor growth was monitored. Eight days later, designated as Day 1 of the study, individual tumor volumes were 63 to 196 mm³, and animals were sorted into eleven groups (n=10) having mean tumor volumes of 114-115 mm³. Tumor volume was calculated using the formula:

$$Tumor Volume ( {mm}^{3} ) = \frac{{w}^{2} \times l}{2}$$

where w=width and l=length in mm of an HCT116 tumor. Tumor weight may be estimated with the assumption that 1 mg is equivalent to 1 mm³ of tumor volume.

c. Therapeutic Agents. Elsamitrucin (provided by Spectrum Pharmaceuticals, Lot No. R21014) was formulated in aqueous succinic acid (1.84 mM) as a 1 mg/mL dosing solution. The 0.5 mg/mL dosing solution was prepared by 2-fold dilution in distilled water. Both solutions were prepared fresh for each dose administered.

Dosing solutions of cisplatin (American Pharmaceutical Partners, Lot No. 728853) were prepared in saline at 0.27 and 0.135 mg/mL. These dosing solutions were prepared once and stored at 4° C. throughout the dosing period.

Stock solutions of 7.5 and 15 mg/mL paclitaxel (Natural Pharmaceuticals, Lot No. 05/208) were prepared in 50% ethanol:50% Cremophor EL and stored at room temperature for the duration of the dosing period. Dosing solutions of 0.75 and 1.5 mg/mL were formulated fresh for each dose by diluting the stock solution ten-fold with D5W.

d. Treatment Plan. A summary of the treatment plan is presented in Table 10. The experiment included an untreated control group, groups treated in monotherapy with two dose levels each of elsamitrucin, paclitaxel or cisplatin, and groups treated with elsamitrucin and paclitaxel or cisplatin combined with the low or the high doses of each agent. Note the routes of administration and schedules detailed in Table 10. All doses were given in volumes of 0.2 mL per 20 g of body weight (10 mL/kg), and were scaled to the body weight of the animal.

e. Endpoint. Tumors were measured twice weekly using calipers. Each animal was euthanized when its tumor reached the endpoint size of 2000 mm³ or at the conclusion of the study on Day 59, whichever came first. The time to endpoint (TTE) for each mouse was calculated from the following equation:

$$TTE ( days ) = \frac{{log}_{10} ( endpoint volume , {mm}^{3} ) - b}{m}$$

where b is the intercept and m is the slope of the line obtained by linear regression of a log-transformed tumor growth data set. The data set was comprised of the first observation that exceeded the study endpoint volume and the three consecutive observations that immediately preceded the attainment of the endpoint volume. Animals that did not reach the endpoint were assigned a TTE value equal to the last day of the study. Animals classified as NTR (non-treatment-related) deaths due to accident (NTRa) or due to unknown causes (NTRu) were excluded from TTE calculations (and all further analyses). Animals classified as TR (treatment-related) deaths or NTRm (non-treatment-related death due to metastasis) were assigned a TTE value equal to the day of death.

Treatment outcome was evaluated by tumor growth delay (TGD), which is defined as the increase in the median time to endpoint (TTE) in a treatment group compared to the control group:

TGD=T−C,

expressed in days, or as a percentage of the median TTE of the control group:

$$\% TGD = \frac{T - C}{C} \times 100$$

where:

T=median TTE for a treatment group, and

C=median TTE for the control group (Group 1).

Treatment may cause partial regression (PR) or complete regression (CR) of the tumor in an animal. In a PR response, the tumor volume is 50% or less of its Day 1 volume for three consecutive measurements during the course of the study, and equal to or greater than 13.5 mm3 for one or more of these three measurements. In a CR response, the tumor volume is less than 13.5 mm3 for three consecutive measurements during the course of the study. An animal with a CR response at the termination of a study is additionally classified as a tumor-free survivor (TFS). Regression responses were monitored and recorded.

f. Toxicity. Animals were weighed daily for the first five days of the study and then twice weekly. The mice were observed frequently for overt signs of any adverse, treatment-related side effects, and clinical signs of toxicity were recorded when observed. Acceptable toxicity is defined as a group mean body-weight loss of less than 20% during the study and not more than one treatment-related (TR) death among ten treated animals. Any dosing regimen that resulted in greater toxicity was considered above the maximum tolerated dose (MTD). A death was classified as TR if attributable to treatment side effects as evidenced by clinical signs and/or necropsy, or may be classified as TR if due to unknown causes during the dosing period or within 10 days of the last dose. A death was classified as an NTR if there is no evidence that death was related to treatment side effects.

g. Statistical and Graphical Analyses. The Logrank test was used to analyze the significance of the differences between the TTE values of treated and control groups. Two-tailed statistical analyses were conducted at significance level P=0.05.

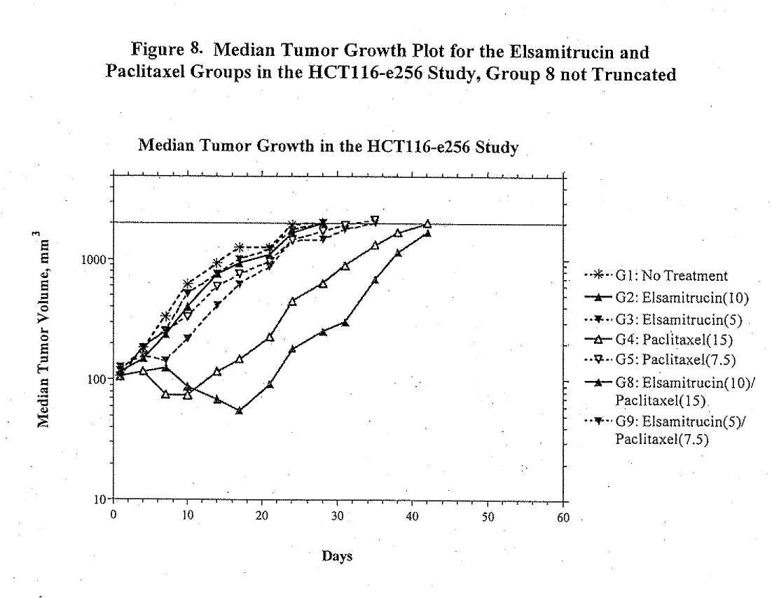

Median tumor growth curves show group median tumor volumes as a function of time. When an animal exited the study due to tumor size, the final tumor volume recorded for the animal was included with the data used to calculate the group median tumor volume at subsequent time points. Curves were truncated after 50% of the animals in a group had exited the study, or after the second TR death in a group, whichever came first (see the exception in FIG. 8). Kaplan-Meier plots were constructed to show the percentage of animals remaining in the study as a function of time. These plots used the same data set as the Logrank test. Prism (GraphPad) for Windows 3.03 was used for all graphic presentations and statistical analyses.

### Results

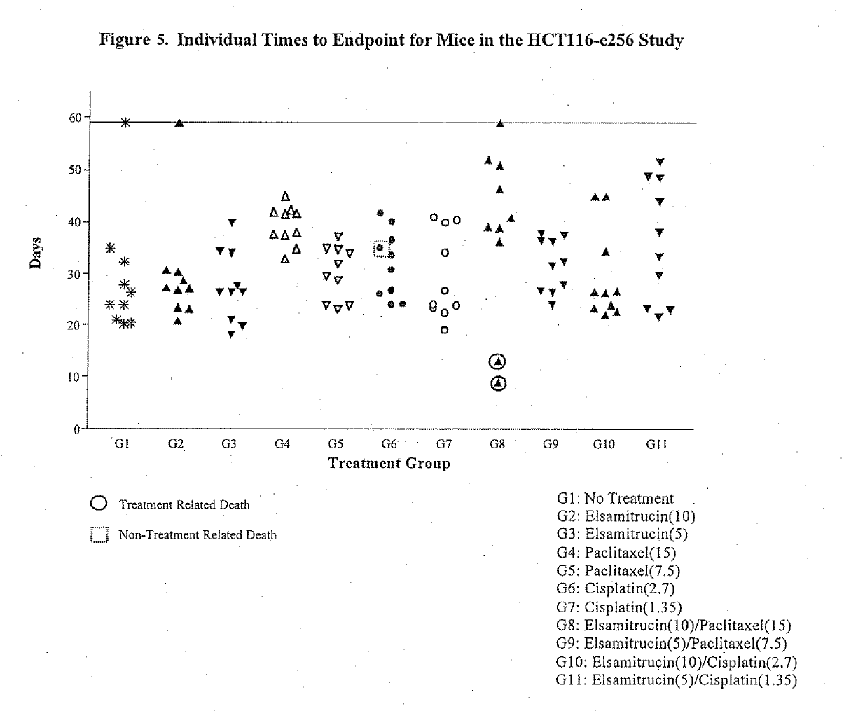

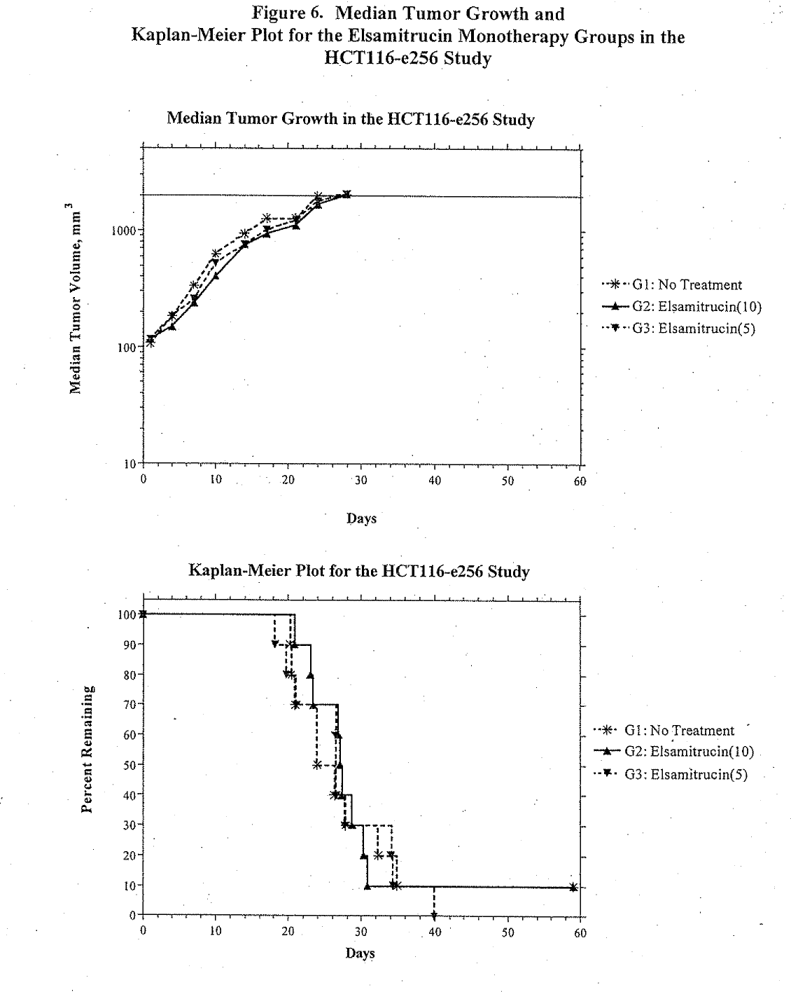

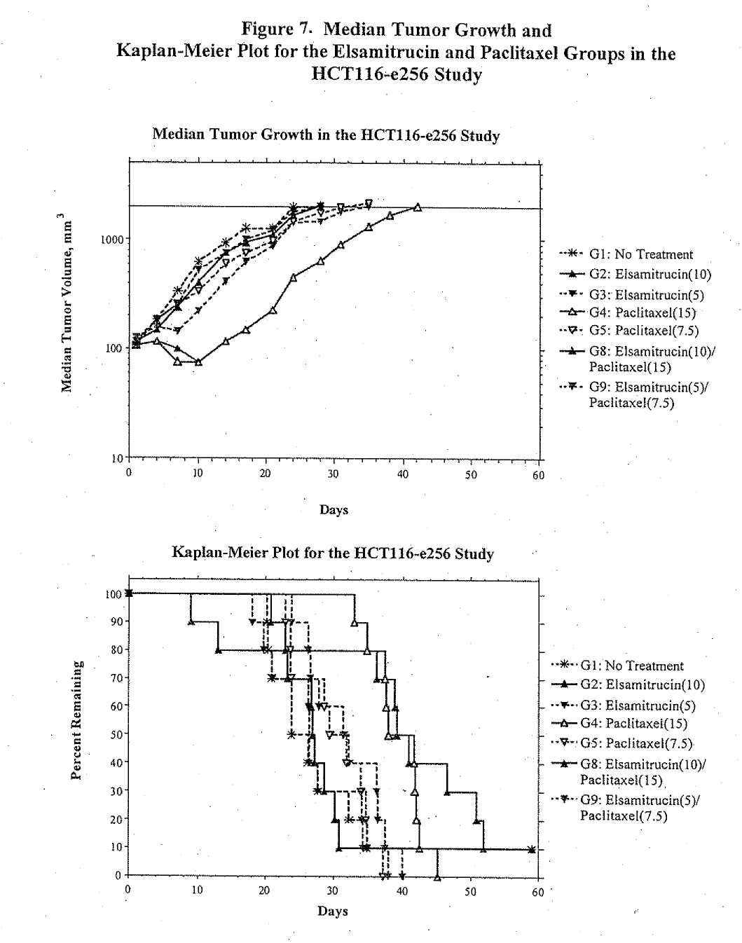

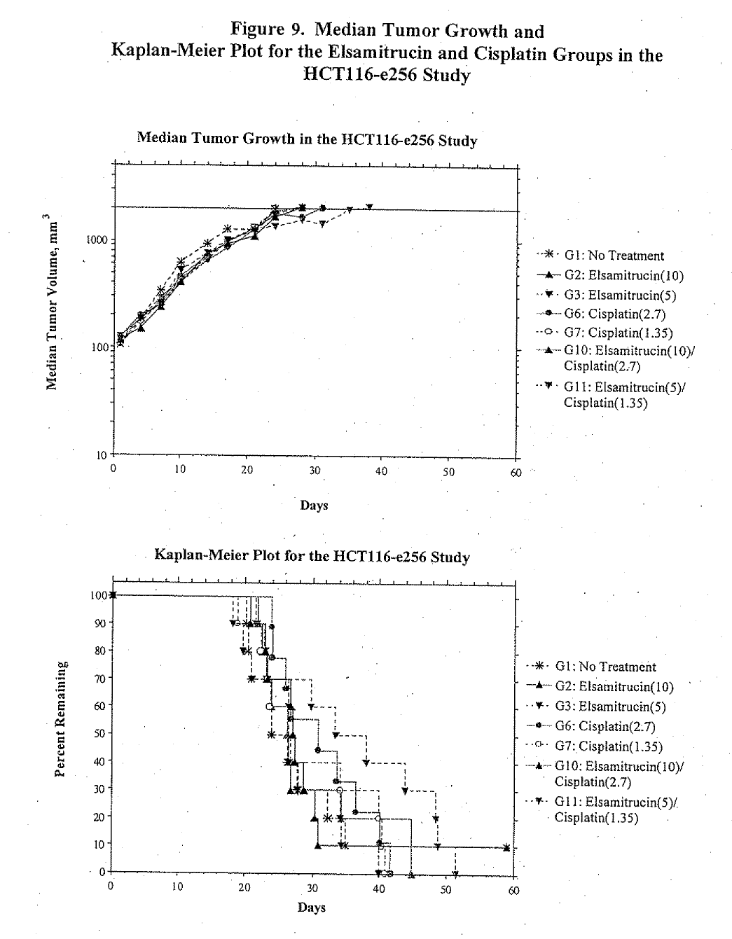

Groups in the HCT116-e256 study were treated in accordance with the protocol in Table 10. Table 11 summarizes the treatment responses for each group. FIG. 5 contains a scatter plot of TTE values by group for each animal in the study. FIGS. 6, 7 and 9 show the group median tumor growth curves (upper panel) and Kaplan-Meier plots (lower panel), respectively, for the groups treated with elsamitrucin monotherapy, for groups administered elsamitrucin and paclitaxel in monotherapy and in combination, and for groups given elsamitrucin and cisplatin in monotherapy and in combination. Note in FIG. 7 that the tumor growth curve for Group 8 was truncated after the second TR death, according to standard test facility statistical practice. FIG. 8 shows the median tumor growth curve for the groups shown in FIG. 7 excluding data for the two TR deaths in Group 8.

a. Efficacy.

i. Growth of HCT116 Tumors in Untreated Mice (Group 1). Tumors (9/10) in untreated mice grew progressively to the 2000 mm³ endpoint. The group had a single animal survive to the end of the study with a complete tumor regression (Table 11 and FIG. 5). The group median TTE was 25.1 days, making the maximum possible TGD for treatment groups 33.9 days, or 135%.

ii. Effect of Treatment with Elsamitrucin Monotherapy (Groups 2 and 3). Tumors in animals treated with elsamitrucin (10 or 5 mg/kg, i.p., q4d ×3) tracked closely with control tumors (FIG. 6), giving group median TTE values of 27.1 and 26.5 days, respectively (Table 10). These values did not represent significant delays in tumor growth relative to the untreated group (P=0.9473 for 10 mg/kg elsamitrucin, P=0.8897 for 5 mg/kg elsamitrucin. No tumor regressions were recorded for the elsamitrucin monotherapy groups, but the group given the 10 mg/kg dose level had a single mouse surviving the study with a tumor measuring 88 mm³.

iii. Effect of Treatment with Elsamitrucin in Combination with Paclitaxel (Groups 4, 5, 8 and 9). Paclitaxel was administered in the present study at dose levels below the optimum therapeutic dose of 30 mg/kg, i.v., qod ×5, identified in previous studies. Treatments at 15 or 7.5 mg/kg, i.v., qod ×5 (Groups 4 and 5, respectively) produced group median TTE values of 39.8 and 30.7 days (Table 11). While tumor growth in these groups appeared to engender dose-related antitumor activity (FIGS. 5 and 7), the median TTE values for neither group achieved statistical significance by logrank analysis (P=0.0578 for 15 mg/kg paclitaxel, P=0.6967 for 7.5 mg/kg). The group given the higher dose level of paclitaxel had 1/10 partial regression response.

The combination of elsamitrucin and paclitaxel at the higher dose levels (10 mg/kg elsamitrucin/15 mg/kg paclitaxel, Group 8) resulted in 2/10 deaths occurring shortly after completion of the dosing period. Necropsies were not performed at the time of these deaths to verify toxicity, but they are considered treatment related by temporal proximity with the dosing regimen. Because of the deaths, the treatment given to Group 8 is considered above the maximum tolerated dose and cannot be evaluated statistically for antitumor activity. However, examination of the individual times to endpoint (FIG. 5) and tumor growth curve (FIG. 8) for the remaining Group 8 animals suggests a positive delay in tumor development produced by this treatment combination.

The combination of elsamitrucin (5 mg/kg) and paclitaxel (7.5 mg/kg) (Group 9) produced a group median TTE of 31.9 days and a TGD of 27%, not significant compared with the untreated animals (P=0.3413).

iv. Effect of Treatment with Elsamitrucin in Combination with Cisplatin (Groups 6, 7, 10 and 11). Cisplatin (2.7 or 1.35 mg/kg, i.p., qd ×5) treatment in monotherapy produced group median TTE values of 30.8 and 25.3 days, respectively (Groups 6 and 7), corresponding to tumor growth delays of 23% and 1%, neither statistically different from the untreated group (P=0.4734 for 2.7 mg/kg cisplatin, P=0.8896 for 1.35 mg/kg). The MTD for cisplatin revealed in previous studies is 2.7 mg/kg, i.p., qd ×5, and the poor antitumor activity seen in Groups 6 and 7 of the present study is consistent with previous experience with this agent in colon models.

Combination of elsamitrucin (10 mg/kg) with cisplatin (2.7 mg/kg) (Group 10) or elsamitrucin (5 mg/kg) with cisplatin (1.35 mg/kg) (Group 11) failed to produce significant tumor growth delays compared with the untreated group (P=0.9123 for Group 10, P=0.8292 for Group 11).

b. Side Effects. Animals were monitored for signs of toxicity by frequent observation and by body weight (BW) measurements (see Table 11 and Clinical Observations). With the exception of Group 8, receiving the combination of the high doses of elsamitrucin and paclitaxel resulting in 2/10 TR deaths, treatments used in the present study were well tolerated. No clinical observations suggesting toxic reactions to the treatment regimens were recorded. Elsamitrucin at the 10 mg/kg dose level produced modest group BW losses, both in monotherapy and in the combinations (Table 11).

### Discussion

The study described in Example 2 evaluated elsamitrucin against the HCT116 human colon carcinoma as monotherapy and in combination with paclitaxel or cisplatin. Tumors in the untreated control mice had a median TTE of 25.1 days with a single animal surviving the study with a tumor regression. With the exception of the group receiving the combination of the high doses of elsamitrucin and paclitaxel, the treatments used in the study were well tolerated. However, all of the treatment regimens tested failed to produce significant delays in tumor growth relative to the untreated group by logrank analysis. It is noteworthy that the dose levels of paclitaxel employed in the present study were below the optimum therapeutic dose of 30 mg/kg identified in previous studies. Further, cisplatin at the doses used in the present study, though at or near the MTD, has shown weak antitumor activity in colon cancer models in the past.

The combination of elsamitrucin (10 mg/kg) and paclitaxel (15 mg/kg) resulted in 2/10 deaths occurring shortly after completion of the dosing period. Although these treatment related deaths rendered the treatment combination nonevaluable by statistical analysis, examination of the individual times to endpoint and tumor growth curve for the remaining animals in the group suggested a delay in tumor development worthy of further evaluation. Exploration of additional dose levels and schedules may uncover an elsamitrucin/paclitaxel combination that is tolerated and efficacious.

Pharmaceutical compositions containing the active ingredients according to the present invention are suitable for administration to humans or other mammals. Typically, the pharmaceutical compositions are sterile, and contain no toxic, carcinogenic, or mutagenic compounds that would cause an adverse reaction when administered. Administration of the pharmaceutical composition can be performed before, during, or after the onset of solid tumor growth.

A method of the present invention can be accomplished using active ingredients as described above, or as a physiologically acceptable salt, derivative, prodrug, or solvate thereof. The active ingredients can be administered as the neat compound, or as a pharmaceutical composition containing either or both entities.

The pharmaceutical compositions include those wherein the active ingredients are administered in an effective amount to achieve their intended purpose. More specifically, a “therapeutically effective amount” means an amount effective to prevent development of, to eliminate, to retard the progression of, or to reduce the size of a solid tumor. Determination of a therapeutically effective amount is well within the capability of those skilled in the art, especially in light of the detailed disclosure provided herein.

A “therapeutically effective dose” refers to that amount of the active ingredients that results in achieving the desired effect. Toxicity and therapeutic efficacy of such active ingredients can be determined by standard pharmaceutical procedures in cell cultures or experimental animals, e.g., determining the LD₅₀ (the dose lethal to 50% of the population) and the ED₅₀ (the dose therapeutically effective in 50% of the population). The dose ratio between toxic and therapeutic effects is the therapeutic index, which is expressed as the ratio between LD₅₀ and ED₅₀. A high therapeutic index is preferred. The data obtained can be used in formulating a range of dosage for use in humans. The dosage of the active ingredients preferably lies within a range of circulating concentrations that include the ED₅₀ with little or no toxicity. The dosage can vary within this range depending upon the dosage form employed, and the route of administration utilized.

The exact formulation and dosage is determined by an individual physician in view of the patient's condition.

The amount of pharmaceutical composition administered can be dependent on the subject being treated, on the subject's weight, the severity of the affliction, the manner of administration, and the judgment of the prescribing physician.

The active ingredients can be administered alone, or in admixture with a pharmaceutical carrier selected with regard to the intended route of administration and standard pharmaceutical practice. Pharmaceutical compositions for use in accordance with the present invention thus can be formulated in a conventional manner using one or more physiologically acceptable carriers comprising excipients and auxiliaries that facilitate processing of the active ingredients into preparations which can be used pharmaceutically.

When a therapeutically effective amount of the active ingredients is administered, the composition can be in the form of a pyrogen-free, parenterally acceptable aqueous solution. The preparation of such parenterally acceptable solutions, having due regard to pH, isotonicity, stability, and the like, is within the skill in the art.

For veterinary use, the active ingredients are administered as a suitably acceptable formulation in accordance with normal veterinary practice. The veterinarian can readily determine the dosing regimen that is most appropriate for a particular animal.

Various adaptations and modifications of the embodiments can be made and used without departing from the scope and spirit of the present invention which can be practiced other than as specifically described herein. The above description is intended to be illustrative, and not restrictive. The scope of the present invention is to be determined only by the claims.

The terms and expressions which have been employed herein are used as terms of description and not of limitation, and there is no intention in the use of such terms and expressions of excluding equivalents of the features shown and described, or portions thereof, it being recognized that various modifications are possible within the scope of the present invention claimed. Moreover, any one or more features of any embodiment of the present invention can be combined with any one or more other features of any other embodiment of the present invention, without departing from the scope of the present invention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the present invention are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the present invention otherwise claimed. No language in the specification should be construed as indicating any non-claimed element essential to the practice of the present invention.

Groupings of alternative elements or embodiments of the present invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Certain embodiments according to the present invention are described herein, including the best mode known to the inventors for carrying out the invention. Of course, variations on these embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the present invention disclosed herein are illustrative of the principles of the present invention. Other modifications that may be employed are within the scope of the present invention. Thus, by way of example, but not of limitation, alternative configurations of the present invention may be utilized in accordance with the teachings herein. Accordingly, the present invention is not limited to that precisely as shown and described.

TABLE 1
Growth Inhibitory Activities of Elasmitrucin and Selected Anticancer Agents against
the HT29 Human Colon Carcinoma Cell Line

| | SPA-01 | | | | | | | | |
|---|---|---|---|---|---|---|---|---|---|
| | (HT29-e21) | | SPA-04 (HT29-e22) | | | SPA-10 (HT29-e24) | | | |
| | | Prism | | Prism | CalcuSyn | | Prism | CalcuSyn | Average |
| | Dilution | IC₅₀ | Dilution | IC₅₀ | Dₘ | Dilution | IC₅₀ | Dₘ | IC_{50,} Dₘ value |
| Elsamitrucin | 1:3 | 0.08 | 1:1.5 | 0.07 | 0.081 | | | | 0.077 |
| 5-Fluorouracil | 1:3 | ~3.3^{a} | 1:1.5 | 0.28 | 0.134^{b} | 1:1.5 | 1.65 | 4.2 | 2.043 |
| Bortezomib | 1:3 | 0.012 | 1:1.5 | 0.023 | 0.029 | | | | 0.021 |
| Camptothecin | 1:3 | 0.016 | 1:1.5 | 0.023 | 0.026 | 1:1.5 | 0.55 | 0.148 | 0.153 |
| Carmustine | 1:3 | >10^{a} | 1:1.5 | 72 | 60 | | | | 66.000 |
| Cisplatin | 1:3 | 1.85 | 1:1.5 | 1.856 | 1.93 | | | | 1.879 |
| Doxorubicin | 1:3 | 0.04 | 1:1.5 | 0.029 | 0.057 | | | | 0.042 |
| Etoposide | 1:3 | ~0.9^{a} | 1:1.5 | 0.698 | 0.766 | | | | 0.732 |
| Gemcitabine | 1:3 | 0.0046 | 1:1.5 | 0.01^{a} | | 1:1.5 | 0.004 | 0.006 | 0.005 |
| Methotrexate | 1:3 | 0.0254 | 1:1.5 | 0.018 | 0.023 | | | | 0.022 |
| Paclitaxel | 1:3 | 0.0016 | 1:1.5 | 0.0024 | 0.0023 | | | | 0.002 |

^{a}Concentration range too low for accurate IC₅₀ determination; value excluded from average.
^{b}Concentration range too high for accurate IC₅₀ determination; value excluded from average.

TABLE 2
Dₘ Values for Elsamitrucin and Selected Anticancer Agents, Individually and in Combinations,
for Growth Inhibition of HT29 Human Colon Carcinoma Cell

| | | | Conc. | | Dₘ Value (95% Confidence Interval), μM | | | |
|---|---|---|---|---|---|---|---|---|
| | | | Range^{a}, | | Individually | | In Combination | |
| Experiment ID | Agent | Ratio | μM | Dilutions | Elsamitrucin | Second Agent | Elsamitrucin | Second Agent |
| SPA-20 HT29-e28 | Elasmitrucin | 1:30 | 0.0176-0.20 | 1.5X | 0.103 | 0.786 | 0.033 | 0.977 |
| (Plate 3) | 5-Fluorouracil | | 0.5267-6 | | (0.053-0.200) | (0.633-0.976) | (0.006-0.175) | (0.182-5.242) |
| SPA-16 HT29-e27 | Elasmitrucin | 1:0.5 | 0.0176-0.20 | 1.5X | 0.036 | 0.0178 | 0.074 | 0.0369 |
| (Plate 3) | Bortezomib | | 0.0088-0.1 | | (0.006-0.237) | (0.0039-0.0819) | (0.034-0.158) | (0.0172-0.0792) |
| SPA-20 HT29-e28 | Elasmitrucin | 1:0.5 | 0.0176-0.2 | 1.5X | 0.118 | 0.02 | 0.038 | 0.019 |
| (Plate 5) | Camptothecin | | 0.0088-0.1 | | (0.070-0.199) | (0.006-0.068) | (0.009-0.160) | (0.005-0.080) |
| SPA-20 HT29-e28 | Elasmitrucin | 1:1800 | 0.0176-0.2 | 1.5X | 0.096 | 128.4 | 0.066 | 118.7 |
| (Plate 2) | Carmustine | | 31.6-360 | | (0.051-0.181) | (120.4-136.9) | (0.018-0.241) | (32.6-433.1) |
| SPA-13 HT29-e26 | Elasmitrucin | 1:46.7 | 0.016-0.09 | 1.33X | 0.058 | 2.328 | 0.031 | 1.434) |
| (Plate 1) | Cisplatin | | 0.74-4.2 | | (0.039-0.086) | (2.223-2.438) | (0.018-0.052) | (0.843-2.441) |
| SPA-13 HT29-e26 | Elsamitrucin | 1:0.6 | 0.016-0.09 | 1.33X | 0.054 | 0.048 | 0.044 | 0.026 |
| (Plate 2) | Doxorubicin | | 0.0096-0.054 | | (0.015-0.186) | (0.013-0.176) | (0.022-0.089) | (0.013-0.053) |
| SPA-07 HT29-e23 | Elsamitrucin | 1:6.25 | 0.0351-0.4 | 1.5X | 0.054 | 0.71 | 0.044 | 0.278 |
| (Plate 3) | Etoposide | | 0.22-2.5 | | (0.034-0.085) | (0.67-0.76) | (0.018-0.112) | (0.110-0.699) |
| SPA-13 HT29-e26 | Elsamitrucin | 1:0.125 | 0.0021-0.08 | 1.25X | 0.067 | 0.004 | 0.027 | 0.0034 |
| (Plate 4) | Gemcitabine | | 0.0026-0.01 | | (0.037-0.121) | (0.0008-0.020) | (0.013-0.056) | (0.0016-0.0070) |
| SPA-07 HT29-e23 | Elsamitrucin | 1:0.2 | 0.0351-0.4 | 1.5X | 0.039 | 0.022 | 0.037 | 0.0073 |
| (Plate 5) | Methotrexate | | 0.007-0.08 | | (0.013-0.116) | (0.0036-0.138) | (0.009-0.157) | (0.0017-0.0311) |
| SPA-07 HT29-e23 | Elsamitrucin | 1:0.03 | 0.0356-0.2 | 1.33X | 0.040 | 0.0032 | 0.043 | 0.0013 |
| (Plate 2) | Paclitaxel | | 0.0011-0.006 | | (0.0076-0.215) | (0.00027-0.0368) | (0.018-0.105) | (0.0005-0.0032) |

^{a}The concentration ranges in the combination are ½ of the concentration of single agent alone

TABLE 2 (Supplement).
Dₘ Values for Elsamitrucin and Selected Anticancer Agents, Individually
and in Combinations, for Growth Inhibidon of HT29 Human Colon Carcinoma Cell

| | | | | | Dₘ Value (95% | |
|---|---|---|---|---|---|---|
| | | | | | Confidence Interval), μM | |
| | | | | | Individually | |
| Experiment ID | Agent | Ratio | Conc. Range^{a}, μM | Dilutions | Elsamitrucin | Second Agent |
| SPA-13 HT29-e26 (Plate 3) | Elasmitrucin | 1:111 | 0.016-0.09 | 1.33X | 0.051 | 2.8 |
| | 5-Fluorouracil | | 1.78-10 | | (0.025-0.104) | (2.37-3.35) |
| SPA-16 HT29-e27 (Plate 5) | Elasmitrucin | 1:70 | 0.0176-0.20 | 1.5X | 0.037 | 0.16 |
| | 5-Fluorouracil | | 1.22-14 | | (0.006-0.225) | (0.077-0.318) |
| SPA-20 HT29-e28 (Plate 3) | Elasmitrucin | 1:30 | 0.0176-0.20 | 1.5X | 0.103 | 0.786 |
| | 5-Fluorouracil | | 0.5267-6 | | (0.053-0.200) | (0.633-0.976) |
| SPA-20 HT29-e28 (Plate 4) | Elasmitrucin | 1:70 | 0.0176-0.20 | 1.5X | na | na |
| | 5-Fluorouracil | | 1.23-14 | | na | na |
| SPA-07 HT29-e23 (Plate 6) | Elasmitrucin | 1:0.2 | 0.0351-0.40 | 1.5X | 0.029 | na |
| | Bortezomib | | 0.007-0.08 | | (0.007-0.114) | na |
| SPA-12 HT29-e25 (Plate 3) | Elasmitrucin | 1:0.113 | 0.016-0.09 | 1.33X | 0.086 | na |
| | Bortezomib | | 0.0021-0.012 | | (0.039-0.191) | na |
| SPA-16 HT29-e27 (Plate 3) | Elasmitrucin | 1:0.5 | 0.0176-0.20 | 1.5X | 0.036 | 0.0178 |
| | Bortezomib | | 0.0088-0.1 | | (0.006-0.237) | (0.0039-0.0819) |
| SPA-12 HT29-e25 (Plate 2) | Elasmitrucin | 1:4 | 0.016-0.09 | 1.33X | 0.073 | 0.24 |
| | Camptothecin | | 0.0641-0.36 | | (0.034-0.155) | (0.148-0.391) |
| SPA-16 HT29-e27 (Plate 2) | Elasmitrucin | 1:3.5 | 0.0176-0.2 | 1.5X | 0.05 | na |
| | Camptothecin | | 0.0615-0.7 | | (0.022-0.110) | na |
| SPA-20 HT29-e28 (Plate 5) | Elasmitrucin | 1:0.5 | 0.0176-0.2 | 1.5X | 0.118 | 0.02 |
| | Camptothecin | | 0.0088-0.1 | | (0.070-0.199) | (0.006-0.068) |
| SPA-20 HT29-e28 (Plate 6) | Elasmitrucin | 1:4 | 0.0176-0.2 | 1.5X | na | na |
| | Camptothecin | | 0.07-0.8 | | na | na |
| SPA-12 HT29-e25 (Plate 1) | Elasmitrucin | 1:1667 | 0.016-0.09 | 1.33X | 0.071 | 123 |
| | Carmustine | | 26.7-150 | | (0.036-0.138) | (109-138) |
| SPA-16 HT29-e27 (Plate 1) | Elasmitrucin | 1:1800 | 0.0176-0.2 | 1.5X | 0.05 | 56.5 |
| | Carmustine | | 31.6-360 | | (0.016-0.160) | (49.5-64.5) |
| SPA-20 HT29-e28 (Plate 1) | Elasmitrucin | 1:1200 | 0.0176-0.2 | 1.5X | 0.124 | 177.4 |
| | Carmustine | | 21.1-240 | | (0.041-0.372) | (154.3-204.0) |
| SPA-20 HT29-e28 (Plate 2) | Elasmitrucin | 1:1800 | 0.0176-0.2 | 1.5X | 0.096 | 128.4 |
| | Carmustine | | 31.6-360 | | (0.051-0.181) | (120.4-136.9) |
| SPA-07 HT29-e23 (Plate 1) | Elasmitrucin | 1:15 | 0.0351-0.40 | 1.5X | 0.032 | 1.75 |
| | Cisplatin | | 0.5267-6.0 | | (0.007-0.146) | (1.65-1.86) |
| SPA-13 HT29-e26 (Plate 1) | Elasmitrucin | 1:46.7 | 0.016-0.09 | 1.33X | 0.058 | 2.328 |
| | Cisplatin | | 0.75-4.2 | | (0.039-0.086) | (2.223-2.438) |
| SPA-07 HT29-e23 (Plate 4) | Elsamitrucin | 1:0.5 | 0.0351-0.4 | 1.5X | 0.051 | 0.022 |
| | Doxorubicin | | 0.0176-0.2 | | (0.027-0.095) | (0.0026-0.190) |
| SPA-13 HT29-e26 (Plate 2) | Elsamitrucin | 1:0.6 | 0.016-0.09 | 1.33X | 0.054 | 0.048 |
| | Doxorubicin | | 0.0096-0.054 | | (0.015-0.186) | (0.013-0.176) |
| SAP-16 HT29-e27 (Plate 4) | Elsamitrucin | 1:0.6 | 0.0176-0.2 | 1.5X | 0.027 | 0.035 |
| | Doxorubicin | | 0.0105-0.12 | | (0.003-0.224) | (0.007-0.172) |
| SPA-07 HT29-e23 (Plate 3) | Elsamitrucin | 1:6.25 | 0.0351-0.4 | 1.5X | 0.054 | 0.71 |
| | Etoposide | | 0.22-2.5 | | (0.034-0.085) | (0.67-0.76) |
| SPA-13 HT29-e26 (Plate 4) | Elsamitrucin | 1:0.125 | 0.0021-0.08 | 1.25X | 0.067 | 0.004 |
| | Gemcitabine | | 0.0026-0.01 | | (0.037-0.121) | (0.0008-0.020) |
| SPA-07 HT29-e23 (Plate 5) | Elsamitrucin | 1:0.2 | 0.0351-0.4 | 1.5X | 0.039 | 0.022 |
| | Methotrexate | | 0.007-0.08 | | (0.013-0.116) | (0.0036-0.138) |
| SPA-07 HT29-e23 (Plate 2) | Elsamitrucin | 1:0.03 | 0.0356-0.2 | 1.33X | 0.04 | 0.0032 |
| | Paclitaxel | | 0.0011-0.006 | | (0.0076-0.215) | (0.00027-0.0368) |

| | Dₘ Value (95% | | |
|---|---|---|---|
| | Confidence Interval), μM | | |
| | In Combination | | |
| Experiment ID | Elsamitrucin | Second Agent | Comment |
| SPA-13 HT29-e26 (Plate 3) | 0.023 | 2.6 | |
| | (0.009-0.059) | (1.00-6.54) | |
| SPA-16 HT29-e27 (Plate 5) | 0.042 | 2.93 | 5-FU concs. need to be lower |
| | (0.009-0.190) | (0.645-13.31) | |
| SPA-20 HT29-e28 (Plate 3) | 0.033 | 0.977 | Presented in Table 2 |
| | (0.006-0.175) | (0.182-5.242) | |
| SPA-20 HT29-e28 (Plate 4) | na | na | 5-FU concs. were too high; did not |
| | na | na | analyze |
| SPA-07 HT29-e23 (Plate 6) | 0.016 | na | Bortezomib concs. need to be lower |
| | (0.002-0.107) | na | |
| SPA-12 HT29-e25 (Plate 3) | 0.137 | na | Wrong bortezomib concs. |
| | (0.018-1.029) | na | |
| SPA-16 HT29-e27 (Plate 3) | 0.074 | 0.0369 | Presented in Table 2 |
| | (0.034-0.158) | (0.0172-0.0792) | |
| SPA-12 HT29-e25 (Plate 2) | 0.05 | 0.202 | |
| | (0.011-0.222) | (0.046-0.887) | |
| SPA-16 HT29-e27 (Plate 2) | 0.021 | na | Camptothecin concs. need to be lower |
| | (0.001-0.730) | na | |
| SPA-20 HT29-e28 (Plate 5) | 0.038 | 0.019 | Presented in Table 2 |
| | (0.009-0.160) | (0.005-0.080) | |
| SPA-20 HT29-e28 (Plate 6) | na | na | Not analyzed. Camptothecin concs. |
| | na | na | were too high |
| SPA-12 HT29-e25 (Plate 1) | 0.057 | 94 | Carmustine concs. need to be higher |
| | (0.013-0.255) | (20.9-424) | |
| SPA-16 HT29-e27 (Plate 1) | 0.092 | 165.5 | |
| | (0.041-0.206) | (73.7-371.4) | |
| SPA-20 HT29-e28 (Plate 1) | 0.102 | 122.7 | |
| | (0.058-0.179) | (70.2-214.5) | |
| SPA-20 HT29-e28 (Plate 2) | 0.066 | 118.7 | Presented in Table 2 |
| | (0.018-0.241) | (32.6-433.1) | |
| SPA-07 HT29-e23 (Plate 1) | 0.034 | 0.504 | |
| | (0.006-0.207) | (0.082-3.11) | |
| SPA-13 HT29-e26 (Plate 1) | 0.031 | 1.434) | Presented in Table 2 |
| | (0.018-0.052) | (0.843-2.441) | |
| SPA-07 HT29-e23 (Plate 4) | 0.040 | 0.020 | |
| | (0.010-0.156) | (0.005-0.078) | |
| SPA-13 HT29-e26 (Plate 2) | 0.044 | 0.026 | Presented in Table 2 |
| | (0.022-0.089) | (0.013-0.053) | |
| SAP-16 HT29-e27 (Plate 4) | 0.131 | 0.078 | |
| | (0.056-0.310) | (0.033-0.186) | |
| SPA-07 HT29-e23 (Plate 3) | 0.044 | 0.278 | Presented in Table 2 |
| | (0.018-0.112) | (0.110-0.699) | |
| SPA-13 HT29-e26 (Plate 4) | 0.027 | 0.0034 | Presented in Table 2 |
| | (0.013-0.056) | (0.0016-0.0070) | |
| SPA-07 HT29-e23 (Plate 5) | 0.037 | 0.0073 | Presented in Table 2 |
| | (0.009-0.157) | (0.0017-0.0311) | |
| SPA-07 HT29-e23 (Plate 2) | 0.043 | 0.0013 | Presented in Table 2 |
| | (0.018-0.105) | (0.0005-0.0032) | |

^{a}The concentration ranges in the combination are ½ of the concentration of single agent alone

TABLE 3
Combination Index Values for Elsamitrucin and Selected Anticancer Agents with the HT29 Human Colon Carcinoma Cell Line

| | | Combination Index Values at 50%, 70%, and 90% Effect Levels | | | | | | | |
|---|---|---|---|---|---|---|---|---|---|
| | | Mutually Exclusive Model | | | | Mutally Non-Exclusive Model | | | |
| Experiment ID | Test Agents | ED₅₀ | ED₇₅ | ED₉₀ | Interactions | ED₅₀ | ED₇₅ | ED₉₀ | Interactions |
| SPA-20 HT29-e28 | Elsamitrucin + 5-Fluorouracil | 1.56 | 1.23 | 0.99 | Antagonism to Nearly | 1.95 | 1.52 | 1.20 | Antagonism to |
| (Plate 3) | | | | | Additive | | | | Moderate Antagonism |
| SPA-16 HT29-e27 | Elasmitrucin + Bortezomib | 4.10 | 3.46 | 3.32 | Strong Antagonism | 8.32 | 6.02 | 4.88 | Strong Antagonism |
| (Plate 3) | | | | | | | | | |
| SPA-20 HT29-e28 | Elsamitrucin + Camptothecin | 1.25 | 1.06 | 0.90 | Moderate antagonism to | 1.55 | 1.24 | 1.01 | Antagonism to |
| (Plate 5) | | | | | Nearly Additive | | | | Nearly Additive |
| SPA-20 HT29-e28 | Elsamitrucin + Carmustine | 1.61 | 2.41 | 3.69 | Antagonism to Strong | 2.24 | 3.73 | 6.44 | Antagonism to |
| (Plate 2) | | | | | Antagonism | | | | Strong Antagonism |
| SPA-13 HT29-e26 | Elsamitrucin + Cisplatin | 1.15 | 1.11 | 1.10 | Slight Antagonism | 1.48 | 1.40 | 1.35 | Antagonism to |
| (Plate 1) | | | | | | | | | Moderate Antagonism |
| SPA-13 HT29-e26 | Elsamitrucin + Doxorubicin | 1.36 | 1.43 | 1.53 | Moderate Antagonism to | 1.80 | 1.94 | 2.12 | Antagonism |
| (Plate 2) | | | | | Antagonism | | | | |
| SPA-07 HT29-e23 | Elsamitrucin + Etoposide | 1.22 | 1.27 | 1.38 | Moderate Antagonism | 1.54 | 1.66 | 1.85 | Antagonism |
| (Plate 3) | | | | | | | | | |
| SPA-13 HT29-e26 | Elasmitrucin + Gemcitabine | 1.36 | 1.42 | 1.53 | Moderate Antagonism to | 1.73 | 1.74 | 1.81 | Antagonism |
| (Plate 4) | | | | | Antagonism | | | | |
| SPA-07 HT29-e23 | Elsamitrucin + Methotrexate | 1.27 | 1.39 | 1.63 | Moderate Antagonism to | 1.57 | 1.84 | 2.29 | Antagonism |
| (Plate 5) | | | | | Antagonism | | | | |
| SPA-07 HT29-e23 | Elsamitrucin + Paclitaxel | 1.47 | 1.27 | 1.26 | Antagonism to Moderate | 1.91 | 1.68 | 1.63 | Antagonism |
| (Plate 2) | | | | | Anatagonism | | | | |

TABLE 3 (Supplement)
Combination Index Value for Elsamitrucin and Selected
Anticancer Agents with the HT29 Human Colon Carcinoma Cell Line

| | | Combination Index Value at | | | |
|---|---|---|---|---|---|
| | | Mutually Exclusive Model | | | |
| Experiment ID | Testing Agents | ED₅₀ | ED₇₅ | ED₉₀ | Interaction |
| SPA-13 HT29-e26 (Plate 3) | Elsamitrucin + 5-Fluorouracil | 1.36 | 1.32 | 1.30 | Moderate Antagonism |
| SPA-16 HT29-e27 (Plate 5) | Elsamitrucin + 5-Fluorouracil | 19.90 | 3.01 | 0.88 | Very Strong Antagonism to |
| | | | | | Slight Synergism |
| SPA-20 HT29-e28 (Plate 3) | Elsamitrucin + 5-Fluorouracil | 1.56 | 1.23 | 0.99 | Antagonism to Nearly |
| | | | | | Additive |
| SPA-20 HT29-e28 (Plate 4) | Elsamitrucin + 5-Fluorouracil | — | — | — | |
| SPA-07 HT29-e23 (Plate 6) | Elsamitrucin + Bortezomib | 1.71 | 1.19 | 0.87 | Antagonism to Slight |
| | | | | | Synergism |
| SPA-12 HT29-e25 (Plate 3) | Elsamitrucin + Bortezomib | — | — | — | |
| SPA-16 HT29-e27 (Plate 3) | Elsamitrucin + Bortezomib | 4.10 | 3.46 | 3.32 | Strong Antagonism |
| SPA-12 HT29-e25 (Plate 2) | Elsamitrucin + Camptothecin | 1.53 | 1.58 | 1.70 | Antagonism |
| SPA-16 HT29-e27 (Plate 2) | Elsamitrucin + Camptothecin | >10 | >10 | >10 | To repeat |
| SPA-20 HT29-e28 (Plate 5) | Elsamitrucin + Camptothecin | 1.15 | 1.06 | 0.90 | Moderate Antagonism to |
| | | | | | Nearly Additive |
| SPA-20 HT29-e28 (Plate 6) | Elsamitrucin + Camptothecin | — | — | — | |
| SPA-12 HT29-e25 (Plate 1) | Elsamitrucin + Carmustine | 1.56 | 1.81 | 2.17 | Antagonism |
| SPA-16 HT29-e27 (Plate 1) | Elsamitrucin + Carmustine | 4.79 | 6.39 | 9.18 | Strong Antagonism |
| SPA-20 HT29-e28 (Plate 1) | Elsamitrucin + Carmustine | 1.51 | 1.76 | 2.16 | Antagonism |
| SPA-20 HT29-e28 (Plate 2) | Elsamitrucin + Carmustine | 1.61 | 2.41 | 3.69 | Antagonism to Strong |
| | | | | | Antagonism |
| SPA-07 HT29-e23 (Plate 1) | Elsamitrucin + Cisplatin | 1.33 | 1.11 | 1.00 | Moderate Antagonism to |
| | | | | | Additive |
| SPA-13 HT29-e26 (Plate 1) | Elsamitrucin + Cisplatin | 1.15 | 1.11 | 1.10 | Slight Antagonism |
| SPA-07 HT29-e23 (Plate 4) | Elsamitrucin + Doxorubicin | 1.69 | 1.54 | 1.41 | Antagonism |
| SPA-13 HT29-e26 (Plate 2) | Elsamitrucin + Doxorubicin | 1.36 | 1.43 | 1.53 | Moderate Antagonism to |
| | | | | | Antagonism |
| SPA-16 HT29-e27 (Plate 4) | Elsamitrucin + Doxorubicin | 7.08 | 7.30 | 8.64 | Strong Antagonism |
| SPA-07 HT29-e23 (Plate 3) | Elsamitrucin + Etoposide | 1.22 | 1.27 | 1.38 | Moderate Antagonism |
| SPA-13 HT29-e26 (Plate 4) | Elsamitrucin + Gemcitabine | 1.36 | 1.42 | 1.53 | Moderate Antagonism to |
| | | | | | Antagonism |
| SPA-07 HT29-e23 (Plate 5) | Elsamitrucin + Methotrexate | 1.27 | 1.39 | 3.63 | Moderate Antagonism to |
| | | | | | Antagonism |
| SPA-07 HT29-e23 (Plate 2) | Elsamitrucin + Paclitaxel | 1.47 | 1.27 | 1.26 | Antagonism to Moderately |
| | | | | | Antagonism |

| | Combination Index Value at | | | | |
|---|---|---|---|---|---|
| | Mutually Non-Exclusive Model | | | | |
| Experiment ID | ED₅₀ | ED₇₅ | ED₉₀ | Interaction | Comment |
| SPA-13 HT29-e26 (Plate 3) | 1.77 | 1.73 | 1.72 | Antagonism | |
| SPA-16 HT29-e27 (Plate 5) | 41.40 | 4.84 | 1.03 | Very Strong Antagonism to | 5-FU concs. need to be lower |
| | | | | Nearly Additive | |
| SPA-20 HT29-e28 (Plate 3) | 1.95 | 1.52 | 1.20 | Antagonism to Moderate | Presented in Table 3 |
| | | | | Antagonism | |
| SPA-20 HT29-e28 (Plate 4) | — | — | — | | 5-FU concs. were too high did |
| | | | | | not analyze |
| SPA-07 HT29-e23 (Plate 6) | 2.35 | 3.45 | 0.97 | Antagonism to Additive | Bortezomib concs. Need to |
| | | | | | be lower |
| SPA-12 HT29-e25 (Plate 3) | — | — | — | | wrong Bortezomib |
| | | | | | concentrations |
| SPA-16 HT29-e27 (Plate 3) | 8.32 | 6.02 | 4.83 | Strong Antagonism | Presented in Table 3 |
| SPA-12 HT29-e25 (Plate 2) | 2.11 | 2.16 | 2.27 | Antagonism | |
| SPA-16 HT29-e27 (Plate 2) | >10 | >10 | >10 | | Camptothecin concs. need |
| | | | | | to be lower |
| SPA-20 HT29-e28 (Plate 5) | 1.55 | 1.24 | 1.01 | Antagonism to Nearly | Presented in Table 3 |
| | | | | Additive | |
| SPA-20 HT29-e28 (Plate 6) | — | — | — | | |
| SPA-12 HT29-e25 (Plate 1) | 2.18 | 2.62 | 3.23 | Antagonism | Carmustine concs. need |
| | | | | | to be higher |
| SPA-16 HT29-e27 (Plate 1) | 10.22 | 14.13 | 20.20 | Very Strong Antagonism | |
| SPA-20 HT29-e28 (Plate 1) | 2.08 | 3.53 | 3.10 | Antagonism | |
| SPA-20 HT29-e28 (Plate 2) | 2.24 | 3.73 | 6.44 | Antagonism to Strong | Presented in Table 3 |
| | | | | Antagonism | |
| SPA-07 HT29-e23 (Plate 1) | 1.63 | 1.39 | 1.24 | Antagonism to Moderate | |
| | | | | Antagonism | |
| SPA-13 HT29-e26 (Plate 1) | 1.48 | 1.40 | 1.35 | Antagonism to Moderate | Presented in Table 3 |
| | | | | Antagonism | |
| SPA-07 HT29-e23 (Plate 4) | 2.40 | 2.13 | 1.90 | Antagonism | |
| SPA-13 HT29-e26 (Plate 2) | 1.80 | 1.94 | 2.12 | Antagonism | Presented in Table 3 |
| SPA-16 HT29-e27 (Plate 4) | 18.00 | 20.60 | 24.86 | Very Strong Antagonism | |
| SPA-07 HT29-e23 (Plate 3) | 1.50 | 1.66 | 1.85 | Antagonism | Presented in Table 3 |
| SPA-13 HT29-e26 (Plate 4) | 1.73 | 1.74 | 1.81 | Antagonism | Presented in Table 3 |
| SPA-07 HT29-e23 (Plate 5) | 1.57 | 1.84 | 2.29 | Antagonism | Presented in Table 3 |
| SPA-07 HT29-e23 (Plate 2) | 1.91 | 1.68 | 1.63 | Antagonism | Presented in Table 3 |

TABLE 4
Growth Inhibitory Activities of Elasmitrucin and Selected Anticancer Agents against
the SKMES Human Non-Small Cell Lung Carcinoma Cell Line

| | SPA-02 (SKMES-e22) | | SPA-03 (SKMES-e23) | | | SPA-09 (SKMES-e25) | | | SPA-14 (SKMES-e27) | | | Average |
|---|---|---|---|---|---|---|---|---|---|---|---|---|
| | | Prism | | Prism | CalcuSyn | | Prism | CalcuSyn | | Prism | CalcuSyn | IC₅₀, |
| | Dilution | IC₅₀ | Dilution | IC₅₀ | Dₘ | Dilution | IC₅₀ | Dₘ | Dilution | IC₅₀ | Dₘ | Dₘ value |
| Elsamitrucin | 1:3 | 0.012 | 1:1.5 | 0.0244 | 0.0256 | | | | | | | 0.021 |
| 5-Fluoruracil | 1:3 | 0.438 | 1:1.5 | ~3.3^{a} | 5.2 | 1:1.5 | 3.13 | 5.58 | | | | 3.587 |
| Bortezomib | | | 1:1.5 | 0.0195 | 0.0215 | | | | | | | 0.021 |
| Camptothecin | 1:3 | 0.006 | 1:1.5 | 0.011 | 0.011 | | | | | | | 0.009 |
| Carmustine | 1:3 | 1.436 | 1:1.5 | na^{c} | na | 1:1.5 | ~78^{a} | ~96^{a} | 1:2 | 9.02 | 8 | 6.152 |
| Carmustine | | | | | | | | | 1:1.5 | 50.25 | 48 | 49.125 |
| Cisplatin | 1:3 | 0.675 | 1:1.5 | 1.142 | 1.185 | | | | | | | 1.001 |
| Doxorubicin | 1:3 | 0.007 | 1:1.5 | ~0.05^{a} | ~0.2^{a} | 1:1.5 | 0.023 | 0.021 | | | | 0.017 |
| Etoposide | 1:3 | 0.199 | 1:1.5 | 0.287 | 0.317 | | | | | | | 0.268 |
| Gemcitabine | 1:3 | 0.0015 | 1:1.5 | 0.0025 | 0.0018 | | | | | | | 0.002 |
| Methotrexate | 1:3 | ~0.0006^{b} | 1:1.5 | na | na | 1:1.5 | 0.0085 | 0.0095 | 1:1.5 | 0.01042 | 0.01069 | 0.010 |
| Paclitaxel | 1:3 | 0.0011 | 1:1.5 | 0.0026 | 0.0028 | | | | | | | 0.002 |

^{a}Concentration range too low for accurate IC50 determination; value excluded from average.
^{b}Concentration range too high for accurate IC50 determination; value excluded from average.
^{c}Not analyzed.

TABLE 5
Dₘ Values for Elsamitrucin and Second Agents, Individually and in Combinations,
for Growth Inhibition of SKMES Human Non-Small Cell Lung Carcinoma Cells

Dₘ Value (95% Confidence Interval), μM

| | | |
|---|---|---|
| Conc. Range^{a}, | Individually | In Combination |

| Experiment ID | Agent | Ratio | μM | Dilutions | Elsamitrucin | Second Agent | Elsamitrucin | Second Agent |
|---|---|---|---|---|---|---|---|---|
| SPA-11 | Elasmitrucin | 1:150 | 0.0107-0.06 | 1.33X | 0.053 | 4.35 | 0.0282 | 4.235 |
| SKMES-e26 | 5-Fluorouracil | | 1.6-9.0 | | (0.001-2.375) | (3.75-5.05) | (0.002-0.368) | (0.325-55.157) |
| (Plate 3) | | | | | | | | |
| SPA-17 | Elasmitrucin | 1:0.67 | 0.0105-0.12 | 1.5X | 0.012 | 0.016 | 0.013 | 0.004 |
| SKMES-e28 | Bortezomib | | 0.007-0.08 | | (0.001-0.101) | (0.008-0.035) | (0.005-0.031) | (0.002-0.010) |
| (Plate 3) | | | | | | | | |
| SPA-06 | Elasmitrucin | 1:0.4 | 0.0107-0.06 | 1.33X | 0.024 | 0.008 | 0.028 | 0.011 |
| SKMES-e24 | Camptothecin | | 0.0043-0.024 | | (0.015-0.040) | (0.002-0.030) | (0.015-0.050) | (0.006-0.020) |
| (Plate 4) | | | | | | | | |
| SPA-17 | Elasmitrucin | 1:1666.7 | 0.0105-0.12 | 1.5X | 0.014 | 69.78 | 0.015 | 24.47 |
| SKMES-e28 | Carmustine | | 17.6-200 | | (0.002-0.133) | (56.27-86.52) | (0.003-0.074) | (4.84-123.72) |
| (Plate 6) | | | | | | | | |
| SPA-06 | Elasmitrucin | 1:50 | 0.0107-0.06 | 1.33X | 0.027 | 1.673 | 0.018 | 0.881 |
| SKMES-e24 | Cisplatin | | 0.53-3.0 | | (0.014-0.052) | 1.616-1.733) | (0.008-0.037) | (0.421-1.844) |
| (Plate 1) | | | | | | | | |
| SPA-17 | Elsamitrucin | 1:1 | 0.0105-0.12 | 1.5X | 0.011 | 0.056 | 0.016 | 0.016 |
| SKMES-e28 | Doxorubicin | | 0.0105-0.12 | | (0.001-0.142) | (0.011-0.291) | (0.002-0.148) | (0.002-0.148) |
| (Plate 1) | | | | | | | | |
| SPA-06 | Elsamitrucin | 1:12 | 0.0107-0.06 | 1.33X | 0.023 | 0.237 | 0.016 | 0.195 |
| SKMES-e24 | Etoposide | | 0.128-0.72 | | (0.010-0.054) | (0.210-0.266) | (0.008-0.0328) | (0.096-0.393) |
| (Plate 3) | | | | | | | | |
| SPA-17 | Elsamitrucin | 1:0.05 | 0.0105-0.12 | 1.5X | 0.013 | 0.0028 | 0.014 | 0.0007 |
| SKMES-e28 | Gemcitabine | | 0.0005-0.006 | | (0.002-0.091) | (0.0000-0.761) | (0.004-0.058) | (0.0002-0.0029) |
| (Plate 4) | | | | | | | | |
| SPA-17 | Elsamitrucin | 1:0.33 | 0.0105-0.12 | 1.5X | 0.014 | 0.0082 | 0.016 | 0.005 |
| SKMES-e28 | Methotrexate | | 0.0035-0.04 | | (0.001-0.138) | (0.0014-0.0487) | (0.003-0.082) | (0.001-0.027) |
| (Plate 2) | | | | | | | | |
| SPA-06 | Elsamitrucin | 1:0.1 | 0.0107-0.06 | 1.33X | 0.027 | 0.0035 | 0.0199 | 0.00199 |
| SKMES-e24 | Paclitaxel | | 0.0011-0.006 | | (0.013-0.053) | (0.00014-0.0861) | (0.0055-0.073) | (0.00055-0.00726) |
| (Plate 2) | | | | | | | | |

^{a}The concentration ranges in the combination are ½ of the concentration of single agent alone

TABLE 5 (Supplement)
Dₘ Values for Elsamitrucin and Second Agents, Individually and in Combinations,
for Growth Inhibition of SKMES Human Non-Small Cell Lung Carcinoma Cell Line

| | | | | | Dₘ Value (95% | |
|---|---|---|---|---|---|---|
| | | | | | Confidence Interval), μM | |
| | | | Conc. Range^{a}, | | Individually | |
| Experiment ID | Agent | Ratio | μM | Dilutions | Elsamitrucin | Second Agent |
| SPA-11 SKMES-e26 (Plate 3) | Elasmitrucin | 1:150 | 0.0107-0.06 | 1.33X | 0.053 | 4.35 |
| | 5-Fluorouracil | | 1.6-9 | | (0.001-2.375) | (3.75-5.05) |
| SPA-06 SKMES-e24 (Plate 6) | Elasmitrucin | 1:0.833 | 0.0107-0.06 | 1.33X | 0.026 | 0.024 |
| | Bortezomib | | 0.0089-0.05 | | (0.018-0.037) | (0.007-0.076) |
| SPA-11 SKMES-e268 (Plate 5) | Elasmitrucin | 1:1 | 0.0131-0.05 | 1.25X | 0.17 | 0.013 |
| | Bortezomib | | 0.0131-0.05 | | (0.006-5.37) | (0.0039-0.046) |
| SPA-17 SKMES-e28 (Plate 3) | Elasmitrucin | 1:0.67 | 0.0105-0.12 | 1.5X | 0.012 | 0.016 |
| | Bortezomib | | 0.007-0.08 | | (0.001-0.101) | (0.008-0.035) |
| SPA-06 SKMES-e24 (Plate 4) | Elasmitrucin | 1:0.4 | 0.0107-0.06 | 1.33X | 0.024 | 0.008 |
| | Camptothecin | | 0.0043-0.024 | | (0.015-0.040) | (0.002-0.030) |
| SPA-17 SKMES-e28 (Plate 5) | Elasmitrucin | 1:416.7 | 0.0105-0.12 | 1.5X | na | na |
| | Carmustine | | 4.4-50 | | na | na |
| SPA-17 SKMES-e28 (Plate 6) | Elasmitrucin | 1:1666.7 | 0.0105-0.12 | 1.5X | 0.014 | 69.78 |
| | Carmustine | | 17.6-200 | | (0.002-0.133) | (56.27-86.52) |
| SPA-06 SKMES-e24 (Plate 1) | Elasmitrucin | 1:50 | 0.0107-0.06 | 1.33X | 0.027 | 1.673 |
| | Cisplatin | | 0.53-3 | | (0.014-0.052) | (1.616-1.733) |
| SPA-11 SKMES-e26 (Plate 1) | Elsamitrucin | 1:0.8 | 0.0107-0.06 | 1.33X | 0.051 | 0.083 |
| | Doxorubicin | | 0.0085-0.048 | | (0.005-0.499) | (0.007-0.976) |
| SPA-17 SKMES-e28 (Plate 1) | Elsamitrucin | 1:1 | 0.0105-0.12 | 1.5X | 0.011 | 0.056 |
| | Doxorubicin | | 0.0105-0.12 | | (0.001-0.142) | (0.011-0.291) |
| SPA-06 SKMES-e24 (Plate 3) | Elsamitrucin | 1:12 | 0.0107-0.06 | 1.33X | 0.023 | 0.237 |
| | Etoposide | | 0.128-0.72 | | (0.010-0.054) | (0.210-0.266) |
| SPA-06 SKMES-e24 (Plate 5) | Elsamitrucin | 1:0.067 | 0.0107-0.06 | 1.33X | 0.024 | 0.0018 |
| | Gemcitabine | | 0.0007-0.004 | | (0.0096-0.059) | (0.00003-0.095) |
| SPA-11 SKMES-e26 (Plate 4) | Elsamitrucin | 1:0.1 | 0.0131-0.05 | 1.25X | 0.161 | 0.0025 |
| | Gemcitabine | | 0.0013-0.005 | | (0.009-2.75) | (0.0011-0.0058) |
| SPA-17 SKMES-e28 (Plate 4)* | Elsamitrucin | 1:0.05 | 0.0105-0.12 | 1.5X | 0.013 | 0.0028 |
| | Gemcitabine | | 0.0005-0.006 | | (0.002-0.091) | (0.0000-0.761) |
| SPA-11 SKMES-e26 (Plate 2) | Elsamitrucin | 1:0.33 | 0.0107-0.06 | 1.33X | 0.048 | 0.012 |
| | Methotrexate | | 0.0036-0.02 | | (0.023-0.103) | (0.003-0.045) |
| SPA-17 SKMES-e28 (Plate 2) | Elsamitrucin | 1:0.33 | 0.0105-0.12 | 1.5X | 0.014 | 0.0082 |
| | Methotrexate | | 0.0035-0.04 | | (0.001-0.138) | (0.0014-0.0487) |
| SPA-06 SKMES-e24 (Plate 2) | Elsamitrucin | 1:0.1 | 0.0107-0.06 | 1.33X | 0.027 | 0.0035 |
| | Paclitaxel | | 0.0011-0.006 | | (0.013-0.053) | (0.00014-0.0861) |

| | Dₘ Value (95% | | |
|---|---|---|---|
| | Confidence Interval), μM | | |
| | In Combination | | |
| Experiment ID | Elsamitrucin | Second Agent | Comment |
| SPA-11 SKMES-e26 (Plate 3) | 0.0282 | 4.235 | Presented in Table 5 |
| | (0.002-0.368) | (0.325-55.157) | |
| SPA-06 SKMES-e24 (Plate 6) | 0.018 | 0.015 | |
| | (0.0038-0.086) | (0.003-0.071) | |
| SPA-11 SKMES-e268 (Plate 5) | 0.014 | 0.014 | Elsamitrucin concs. |
| | (0.0039-0.0509) | (0.0039-0.0509) | Need to be higher |
| SPA-17 SKMES-e28 (Plate 3) | 0.013 | 0.004 | Presented in Table 5 |
| | (0.005-0.031) | (0.002-0.010) | |
| SPA-06 SKMES-e24 (Plate 4) | 0.028 | 0.011 | Presented in Table 5 |
| | (0.015-0.050) | (0.006-0.020) | |
| SPA-17 SKMES-e28 (Plate 5) | na | na | Carmustine concentration |
| | na | na | range is not appropriate |
| SPA-17 SKMES-e28 (Plate 6) | 0.015 | 24.47 | Presented in Table 5 |
| | (0.003-0.074) | (4.84-123.72) | |
| SPA-06 SKMES-e24 (Plate 1) | 0.018 | 0.881 | Presented in Table 5 |
| | (0.008-0.037) | (0.421-1.844) | |
| SPA-11 SKMES-e26 (Plate 1) | 0.100 | 0.08 | |
| | (0.0016-6.175) | (0.0013-4.94) | |
| SPA-17 SKMES-e28 (Plate 1) | 0.016 | 0.016 | Presented in Table 5 |
| | (0.002-0.148) | (0.002-0.148) | |
| SPA-06 SKMES-e24 (Plate 3) | 0.016 | 0.195 | Presented in Table 5 |
| | (0.008-0.0328) | (0.096-0.393) | |
| SPA-06 SKMES-e24 (Plate 5) | 0.023 | 0.0015 | |
| | (0.0012-0.438) | (0.00007-0.029) | |
| SPA-11 SKMES-e26 (Plate 4) | 0.037 | 0.0038 | Elsamitrucin concs. |
| | (0.0022-0.0654) | (0.0002-0.0654) | Need to be higher |
| SPA-17 SKMES-e28 (Plate 4)* | 0.014 | 0.0007 | Presented in Table 5 |
| | (0.004-0.058) | (0.0002-0.0029) | |
| SPA-11 SKMES-e26 (Plate 2) | 0.043 | 0.0143 | |
| | (0.0017-1.087) | (0.00056-0.36) | |
| SPA-17 SKMES-e28 (Plate 2) | 0.016 | 0.005 | Presented in Table 5 |
| | (0.003-0.082) | (0.001-0.027) | |
| SPA-06 SKMES-e24 (Plate 2) | 0.0199 | 0.00199 | Presented in Table 5 |
| | (0.0055-0.073) | (0.00055-0.00726) | |

^{a}The concentration ranges in the combination are ½ of the concentration of single agent alone

TABLE 6
Combination Index Values for Elsamitrucin and Selected Anticancer Agents with the SKMES Human Non-Small Cell Lung Carcinoma Cell Line

| | | Calculated Combination Index Values at 50%, 70%, and 90% Effect Levels | | | | | | | |
|---|---|---|---|---|---|---|---|---|---|
| | | Mutually Exclusive Model | | | | Mutally Non-Exclusive Model | | | |
| Experiment ID | Test Agents | ED₅₀ | ED₇₅ | ED₉₀ | Interactions | ED₅₀ | ED₇₅ | ED₉₀ | Interactions |
| SPA-11 SKMES-e26 | Elsamitrucin + 5-Fluorouracil | 1.51 | 1.43 | 1.35 | Antagonism to Moderate | 2.03 | 1.90 | 1.78 | Antagonism |
| (Plate 3) | | | | | Antagonism | | | | |
| SPA-17 SKMES-e28 | Elasmitrucin + Bortezomib | 1.33 | 0.98 | 0.75 | Moderate Antagonism to | 1.61 | 1.17 | 0.87 | Antagonism to Slight |
| (Plate 3) | | | | | Moderate Synergism | | | | Synergism |
| SPA-06 SKMES-e24 | Elsamitrucin + Camptothecin | 2.50 | 2.60 | 2.70 | Antagonism | 4.06 | 4.28 | 4.52 | Strong Antagonism |
| (Plate 4) | | | | | | | | | |
| SPA-17 SKMES-e28 | Elsamitrucin + Carmustine | 1.39 | 1.19 | 1.02 | Moderate Antagonism to | 1.75 | 1.46 | 1.22 | Antagonism to Moderate |
| (Plate 3) | | | | | Nearly Additive | | | | Antagonism |
| SPA-06 SKMES-e24 | Elsamitrucin + Cisplatin | 1.18 | 1.11 | 1.04 | Slight Antagonism to | 1.52 | 1.41 | 1.30 | Antagonism to Moderate |
| (Plate 1) | | | | | Nearly Additive | | | | Antagonism |
| SPA-17 SKMES-e28 | Elsamitrucin + Doxorubicin | 1.75 | 1.22 | 0.85 | Antagonism to Slight | 2.17 | 1.43 | 0.95 | Antagonism to Nearly |
| (Plate 1) | | | | | Synergism | | | | Additive |
| SPA-06 SKMES-e24 | Elsamitrucin + Etoposide | 1.52 | 1.50 | 1.49 | Antagonism | 2.10 | 2.10 | 2.10 | Antagonism |
| (Plate 3) | | | | | | | | | |
| SPA-17 SKMES-e28 | Elsamitrucin + Gemcitabine | 1.40 | 1.20 | 1.03 | Moderate Antagonism to | 1.70 | 1.42 | 1.19 | Antagonism to Slight |
| (Plate 4) | | | | | Nearly Additive | | | | Antagonism |
| SPA-17 SKMES-e28 | Elasmitrucin + Methotrexate | 1.81 | 1.56 | 1.35 | Antagonism to Moderate | 2.57 | 2.13 | 1.78 | Antagonism |
| (Plate 2) | | | | | Antagonism | | | | |
| SPA-06 SKMES-e24 | Elsamitrucin + Paclitaxel | 1.32 | 1.38 | 1.45 | Moderate Antagonism | 1.74 | 1.85 | 1.98 | Antagonism |
| (Plate 2) | | | | | | | | | |

TABLE 6 (Supplement).
Combination Index Value for Elsamitrucin and Selected Anticancer Agents
with the SKMES Human Non-Small Cell Lung Carcinoma Cell Line

| | | Combination Index Value at | | | |
|---|---|---|---|---|---|
| | | Mutually Exclusive Model | | | |
| Experiment ID | Testing Agents | ED₅₀ | ED₇₅ | ED₉₀ | Summary |
| SPA-11 SKMES-e26 (Plate 3) | Elsamitrucin + 5-Fluorouracil | 1.51 | 1.43 | 1.35 | Antagonism to Moderate |
| | | | | | Antagonism |
| SPA-06 SKMES-e24 (Plate 6) | Elsamitrucin + Bortezomib | 1.33 | 1.35 | 1.38 | Moderate Antagonism |
| SPA-11 SKMES-e26 (Plate 5) | Elsamitrucin + Bortezomib | 1.61 | 1.46 | 1.35 | Antagonism to Moderate |
| | | | | | Antagonism |
| SPA-17 SKMES-e28 (Plate 3) | Elasmitrucin + Bortezomib | 1.33 | 0.98 | 0.75 | Moderate Antagonism to |
| | | | | | Moderate Synergism |
| SPA-06 SKMES-e24 (Plate 4) | Elsamitrucin + Camptothecin | 2.50 | 2.60 | 2.70 | Antagonism |
| SPA-17 SKMES-e28 (Plate 5) | Elsamitrucin + Carmustine | — | — | — | |
| SPA-17 SKMES-e28 (Plate 6) | Elsamitrucin + Carmustine | 1.39 | 1.19 | 1.02 | Moderate Antagonism to |
| | | | | | Nearly Additive |
| SPA-06 SKMES-e24 (Plate 1) | Elsamitrucin + Cisplatin | 1.18 | 1.11 | 1.04 | Slight Antagonism to |
| | | | | | Nearly Additive |
| SPA-11 SKMES-e26 (Plate 1) | Elsamitrucin + Doxorubicin | 2.94 | 5.77 | 11.50 | Antagonism to Strong |
| | | | | | Antagonism |
| SPA-17 SKMES-e28 (Plate 1) | Elsamitrucin + Doxorubicin | 1.75 | 1.22 | 0.85 | Antagonism to Slight |
| | | | | | Synergism |
| SPA-06 SKMES-e24 (Plate 3) | Elsamitrucin + Etoposide | 1.52 | 1.50 | 1.49 | Antagonism |
| SPA-06 SKMES-e24 (Plate 5) | Elsamitrucin + Gemcitabine | 1.80 | 2.05 | 2.33 | Antagonism |
| SPA-11 SKMES-e26 (Plate 4) | Elsamitrucin + Gemcitabine | 2.20 | 2.86 | 3.82 | Antagonism to Strong |
| | | | | | Antagonism |
| SPA-17 SKMES-e28 (Plate 4) | Elsamitrucin + Gemcitabine | 1.40 | 1.20 | 1.03 | Moderate Antagonism to |
| | | | | | Nearly Additive |
| SPA-11 SKMES-e26 (Plate 2) | Elsamitrucin + Methotrexate | 2.09 | 2.53 | 3.06 | Antagonism |
| SPA-17 SKMES-e28 (Plate 2) | Elasmitrucin + Methotrexate | 1.81 | 1.56 | 1.35 | Antagonism to Moderate |
| | | | | | Antagonism |
| SPA-06 SKMES-e24 (Plate 2) | Elsamitrucin + Paclitaxel | 1.32 | 1.38 | 1.45 | Moderate Antagonism |

| | Combination Index Value at | | | | |
|---|---|---|---|---|---|
| | Mutally Non-Exclusive Model | | | | |
| Experiment ID | ED₅₀ | ED₇₅ | ED₉₀ | Summary | Comment |
| SPA-11 SKMES-e26 (Plate 3) | 2.03 | 1.90 | 1.78 | Antagonism | Presented in Table 6 |
| SPA-06 SKMES-e24 (Plate 6) | 1.78 | 1.81 | 1.84 | Antagonism | |
| SPA-11 SKMES-e26 (Plate 5) | 1.71 | 1.49 | 1.36 | Antagonism to Moderate | Elsamitrucin concs need to be |
| | | | | Antagonism | higher |
| SPA-17 SKMES-e28 (Plate 3) | 1.61 | 1.17 | 0.87 | Antagonism to Slight | Presented in Table 6 |
| | | | | Synergism | |
| SPA-06 SKMES-e24 (Plate 4) | 4.06 | 4.28 | 4.52 | Strong Antagonism | Presented in Table 6 |
| SPA-17 SKMES-e28 (Plate 5) | — | — | — | | Carmustine concentration |
| | | | | | range is not appropriate |
| SPA-17 SKMES-e28 (Plate 6) | 1.75 | 1.46 | 1.22 | Antagonism to Moderate | Presented in Table 6 |
| | | | | Antagonism | |
| SPA-06 SKMES-e24 (Plate 1) | 1.52 | 1.41 | 1.30 | Antagonism to Moderate | Presented in Table 6 |
| | | | | Antagonism | |
| SPA-11 SKMES-e26 (Plate 1) | 4.83 | 12.43 | 34.83 | Strong Antagonism to Very | |
| | | | | Strong Antagonism | |
| SPA-17 SKMES-e28 (Plate 1) | 2.17 | 1.43 | 0.95 | Antagonism to Nearly | Presented in Table 6 |
| | | | | Additive | |
| SPA-06 SKMES-e24 (Plate 3) | 2.10 | 2.10 | 2.10 | Antagonism | Presented in Table 6 |
| SPA-06 SKMES-e24 (Plate 5) | 2.60 | 3.09 | 3.69 | Antagonism | |
| | | | | to Strong Antagonism | |
| SPA-11 SKMES-e26 (Plate 4) | 2.59 | 3.16 | 4.06 | Antagonism to Strong | Elsamitrucin concs need to be |
| | | | | Antagonism | higher |
| SPA-17 SKMES-e28 (Plate 4) | 1.70 | 1.42 | 1.19 | Antagonism to Slight | Presented in Table 6 |
| | | | | Antagonism | |
| SPA-11 SKMES-e26 (Plate 2) | 3.15 | 4.09 | 5.34 | Antagonism to Strong | |
| | | | | Antagonism | |
| SPA-17 SKMES-e28 (Plate 2) | 2.57 | 2.13 | 1.78 | Antagonism | Presented in Table 6 |
| SPA-06 SKMES-e24 (Plate 2) | 1.74 | 1.85 | 1.98 | Antagonism | Presented in Table 6 |

TABLE 7
Growth Inhibitory Activities of Elasmitrucin and Selected Anticancer Agents against
the Daudi Human Lymphoma Cell Line

| | SPA-05 | | | | | | | | |
|---|---|---|---|---|---|---|---|---|---|
| | (Daudi-e01) | | SPA-08 (Daudi-e02) | | | SPA-15 (Daudi-e03) | | | |
| | | Prism | | Prism | CalcuSyn | | Prism | CalcuSyn | Average |
| | Dilution | IC₅₀ | Dilution | IC₅₀ | Dₘ | Dilution | IC₅₀ | Dₘ | IC₅₀, Dₘ value |
| Elsamitrucin | 1:3 | 0.012 | 1:1.5 | na^{a,c} | na^{a} | 1:1.33 | 0.04 | 0.046 | 0.033 |
| 5-Fluorouracil | 1:3 | ~9.2^{a} | 1:1.5 | 12.7 | 11.12 | 1:1.5 | 11.88 | 13.2 | 12.225 |
| Bortezomib | 1:3 | 0.0037 | 1:1.5 | 0.004 | 0.0034 | | | | 0.004 |
| Camptothecin | 1:3 | 0.0085 | 1:1.5 | 0.0095 | 0.011 | | | | 0.010 |
| Carmustine | 1:3 | na^{a,c} | 1:1.5 | 28 | 25 | 1:1.5 | 20.8 | 25.84 | 24.910 |
| Cisplatin | 1:3 | 0.367 | 1:1.5 | 0.344 | 0.343 | | | | 0.351 |
| Doxorubicin | 1:3 | ~0.001^{b} | 1:1.5 | ~0.0095^{a} | ~0.033^{a} | 1:1.5 | 0.005 | 0.005 | 0.005 |
| Etoposide | 1:3 | 0.0202 | 1:1.5 | 0.031 | 0.023 | | | | 0.025 |
| Gemcitabine | 1:3 | 0.0006 | 1:1.5 | 0.0014 | 0.0013 | | | | 0.001 |
| Methotrexate | 1:3 | ~0.02^{b} | 1:1.5 | 0.049 | 0.050 | 1:1.5 | na^{a} | na^{a} | 0.050 |
| Paclitaxel | 1:3 | 0.0036 | 1:1.5 | 0.0033 | 0.0041 | | | | 0.004 |

^{a}Concentration range too low for accurate IC₅₀ determination; value excluded from average.
^{b}Concentration range too high for accurate IC₅₀ determination; value excluded from average.
^{c}Not analyzed.

TABLE 8
Dₘ Values for Elsamitrucin and Second Agents, Individually and in Combinations,
for Growth Inhibition of Daudi Human Lymphoma Cells

| | | | Conc. | | Dₘ Value (95% Confidence Interval), μM | | | |
|---|---|---|---|---|---|---|---|---|
| | | | Range^{a}, | Dilu- | Individually | | In Combination | |
| Experiment ID | Agent | Ratio | μM | tions | Elsamitrucin | Second Agent | Elsamitrucin | Second Agent |
| SPA-18 Daudi-e04 | Elasmitrucin | 1:333.3 | 0.0105-0.12 | 1.5X | 0.023 | 11.67 | 0.018 | 5.91 |
| (Plate 1) | 5- | | 3.51-40.0 | | (0.006-0.084) | (11.19-14.33) | (0.005-0.058) | (1.82-19.17) |
| | Fluorouracil | | | | | | | |
| SPA-18 Daudi-e04 | Elasmitrucin | 1:0.125 | 0.0105-0.12 | 1.5X | 0.030 | 0.0039 | 0.019 | 0.0023 |
| (Plate 2) | Bortezomib | | 0.0013-0.015 | | (0.006-0.148) | (0.0002-0.0902) | (0.004-0.078) | (0.0006-0.0097) |
| SPA-18 Daudi-e04 | Elasmitrucin | 1:0.33 | 0.0105-0.12 | 1.5X | 0.028 | 0.0093 | 0.016 | 0.0054 |
| (Plate 3) | Camp- | | 0.0035-0.04 | | (0.010-0.078) | (0.0049-0.0176) | (0.007-0.039) | (0.0022-0.0129) |
| | tothecin | | | | | | | |
| SPA-21 Daudi-e06 | Elasmitrucin | 1:1750 | 0.0088-0.10 | 1.5X | 0.013 | 42.0 | 0.012 | 21.5 |
| (Plate 2) | Carmustine | | 15.4-175 | | (0.002-0.087) | (39.8-44.3) | (0.006-0.027) | (9.6-48.0) |
| SPA-18 Daudi-e04 | Elasmitrucin | 1:10.83 | 0.0105-0.12 | 1.5X | 0.034 | 0.494 | 0.019 | 0.203 |
| (Plate 5) | Cisplatin | | 0.114-1.3 | | (0.005-0.249) | (0.372-0.656) | (0.008-0.042) | (0.091-0.452) |
| SPA-18 Daudi-e04 | Elsamitrucin | 1:0.129 | 0.0105-0.12 | 1.5X | 0.026 | 0.009 | 0.020 | 0.0026 |
| (Plate 6) | Doxorubicin | | 0.0014-0.0155 | | (0.007-0.100) | (0.0001-0.834) | (0.010-0.042) | (0.0013-0.0055) |
| SPA-19 Daudi-e05 | Elsamitrucin | 1:0.83 | 0.0105-0.12 | 1.5X | 0.027 | 0.047 | 0.019 | 0.016 |
| (Plate 1) | Etoposide | | 0.0088-0.10 | | (0.008-0.087) | (0.009-0.242) | (0.0097-0.038) | (0.008-0.032) |
| SPA-19 Daudi-e05 | Elsamitrucin | 1:0.033 | 0.0105-0.12 | 1.5X | 0.026 | 0.0012 | 0.018 | 0.0006 |
| (Plate 2) | Gemcitabine | | 0.0004-0.004 | | (0.007-0.091) | (0.00003-0.053) | (0.007-0.045) | (0.00023-0.0015) |
| SPA-19 Daudi-e05 | Elsamitrucin | 1:1.67 | 0.0105-0.12 | 1.5X | 0.026 | 0.045 | 0.021 | 0.036 |
| (Plate 3) | Methotrexate | | 0.0176-0.20 | | (0.007-0.100) | (0.004-0.521) | (0.009-0.051) | (0.015-0.084) |
| SPA-19 Daudi-e05 | Elsamitrucin | 1:0.125 | 0.0105-0.12 | 1.5X | 0.030 | 0.004 | 0.019 | 0.0024 |
| (Plate 4) | Paclitaxel | | 0.0013-0.015 | | (0.006-0.153) | (0.0002-0.0743) | (0.003-0.132) | (0.00036-0.01644) |

^{a}The concentration ranges in the combination are ½ of the concentration of single agent alone

TABLE 8 (Supplement).
Dₘ Values for Elsamitrucin and Second Agents, Individually and in Combinations,
for Growth Inhibition of Daudi Human Lymphoma Cells

| | | | | | Dₘ Value (95% | |
|---|---|---|---|---|---|---|
| | | | Conc. | | Confidence Interval), μM | |
| | | | Range^{a}, | | Individually | |
| Experiment ID | Agent | Ratio | μM | Dilutions | Elsamitrucin | Second Agent |
| SPA-18 Daudi-e04 (Plate 1) | Elasmitrucin | 1:333.3 | 0.0105-0.12 | 1.5X | 0.023 | 11.67 |
| | 5-Fluorouracil | | 3.51-40.0 | | (0.006-0.084) | (11.19-14.33) |
| SPA-18 Daudi-e04 (Plate 2) | Elasmitrucin | 1:0.125 | 0.0105-0.12 | 1.5X | 0.03 | 0.0039 |
| | Bortezomib | | 0.0013-0.0 | | (0.006-0.148) | (0.0002-0.0902) |
| SPA-18 Daudi-e04 (Plate 3) | Elasmitrucin | 1:0.33 | 0.0105-0.12 | 1.5X | 0.028 | 0.0093 |
| | Camptothecin | | 0.0035-0.0 | | (0.010-0.078) | (0.0049-0.0176) |
| SPA-18 Daudi-e04 (Plate 4) | Elasmitrucin | 1:750 | 0.0105-0.12 | 1.5X | 0.03 | 452.0 |
| | Carmustine | | 7.9-90 | | (0.007-0.122) | (45-4522) |
| SPA-21 Daudi-e06 (Plate 1) | Elasmitrucin | 1:1000 | 0.0088-0.10 | 1.5X | 0.015 | 42.7 |
| | Carmustine | | 8.8-100 | | (0.002-0.115) | (34.6-52.7) |
| SPA-21 Daudi-e06 (Plate 2) | Elasmitrucin | 1:1750 | 0.0088-0.10 | 1.5X | 0.013 | 42 |
| | Carmustine | | 15.4-175.0 | | (0.002-0.087) | (39.8-44.3) |
| SPA-21 Daudi-e06 (Plate 3) | Elasmitrucin | 1:2500 | 0.0088-0.10 | 1.5X | 0.012 | 30.2 |
| | Carmustine | | 21.9-250 | | (0.001-0.136) | (21.8-41.8) |
| SPA-18 Daudi-e04 (Plate 5) | Elasmitrucin | 1:10.83 | 0.0105-0.12 | 1.5X | 0.034 | 0.494 |
| | Cisplatin | | 0.114-1.3 | | (0.005-0.249) | (0.372-0.656) |
| SPA-18 Daudi-e04 (Plate 6) | Elsamitrucin | 1:0.129 | 0.0105-0.12 | 1.5X | 0.026 | 0.009 |
| | Doxorubicin | | 0.0014-0.0 | | (0.007-0.100) | (0.0001-0.834) |
| SPA-19 Daudi-e05 (Plate 1) | Elsamitrucin | 1:0.83 | 0.0105-0.12 | 1.5X | 0.027 | 0.047 |
| | Etoposide | | 0.0088-0.1 | | (0.008-0.087) | (0.009-0.242) |
| SPA-19 Daudi-e05 (Plate 2) | Elsamitrucin | 1:0.033 | 0.0105-0.12 | 1.5X | 0.026 | 0.0012 |
| | Gemcitabine | | 0.0004-0.0 | | (0.007-0.091) | (0.00003-0.053) |
| SPA-19 Daudi-e05 (Plate 3) | Elsamitrucin | 1:1.67 | 0.0105-0.12 | 1.5X | 0.026 | 0.045 |
| | Methotrexate | | 0.0176-0.2 | | (0.007-0.100) | (0.004-0.521) |
| SPA-19 Daudi-e05 (Plate 4) | Elsamitrucin | 1:0.125 | 0.0105-0.12 | 1.5X | 0.03 | 0.004 |
| | Paclitaxel | | 0.0013-0.0 | | (0.006-0.153) | (0.0002-0.0743) |

| | Dₘ Value (95% | | |
|---|---|---|---|
| | Confidence Interval), μM | | |
| | In Combination | | |
| Experiment ID | Elsamitrucin | Second Agent | Comment |
| SPA-18 Daudi-e04 (Plate 1) | 0.018 | 5.91 | Presented in Table 8 |
| | (0.005-0.058) | (1.82-19.17) | |
| SPA-18 Daudi-e04 (Plate 2) | 0.019 | 0.0023 | Presented in Table 8 |
| | (0.004-0.078) | (0.0006-0.0097) | |
| SPA-18 Daudi-e04 (Plate 3) | 0.016 | 0.0054 | Presented in Table 8 |
| | (0.007-0.039) | (0.0022-0.0129) | |
| SPA-18 Daudi-e04 (Plate 4) | 0.029 | 21.99 | Carmustine solutions may |
| | (0.013-0.063) | (10.23-47.28) | have not prepared properly |
| SPA-21 Daudi-e06 (Plate 1) | 0.011 | 14.2 | |
| | (0.009-0.023) | (8.7-23.2) | |
| SPA-21 Daudi-e06 (Plate 2) | 0.012 | 21.5 | Presented in Table 8 |
| | (0.006-0.027) | (9.6-48.0) | |
| SPA-21 Daudi-e06 (Plate 3) | 0.011 | 27 | Carmustine concentration |
| | (0.003-0.036) | (8.2-89.6) | range was slightly too high |
| SPA-18 Daudi-e04 (Plate 5) | 0.019 | 0.203 | Presented in Table 8 |
| | (0.008-0.042) | (0.091-0.452) | |
| SPA-18 Daudi-e04 (Plate 6) | 0.02 | 0.0026 | Presented in Table 8 |
| | (0.010-0.042) | (0.0013-0.0055) | |
| SPA-19 Daudi-e05 (Plate 1) | 0.019 | 0.016 | Presented in Table 8 |
| | (0.0097-0.038) | (0.008-0.032) | |
| SPA-19 Daudi-e05 (Plate 2) | 0.018 | 0.0006 | Presented in Table 8 |
| | (0.007-0.045) | (0.00023-0.0015) | |
| SPA-19 Daudi-e05 (Plate 3) | 0.021 | 0.036 | Presented in Table 8 |
| | (0.009-0.051) | (0.015-0.084) | |
| SPA-19 Daudi-e05 (Plate 4) | 0.019 | 0.0024 | Presented in Table 8 |
| | (0.003-0.132) | (0.00036-0.01644) | |

^{a}The concentration ranges in the combination are ½ of the concentration of single agent alone

TABLE 9
Combination Index Values for Elsamitrucin and Selected Anticancer Agents with the Daudi Human Lymphoma Cell Line

| | | Calculated Combination Index Values at 50%, 70%, and 90% Effect Levels | | | | | | | |
|---|---|---|---|---|---|---|---|---|---|
| | | Mutually Exclusive Model | | | | Mutually Non-Exclusive Model | | | |
| Experiment ID | Test Agents | ED₅₀ | ED₇₅ | ED₉₀ | Interactions | ED₅₀ | ED₇₅ | ED₉₀ | Interactions |
| SPA-18 Daudi-e04 | Elsamitrucin + 5-Fluorouracil | 1.26 | 1.17 | 1.10 | Moderate Antagonism to | 1.62 | 1.47 | 1.35 | Antagonism to Moderate |
| (Plate 1) | | | | | Slight Antagonism | | | | Antagonism |
| SPA-18 Daudi-e04 | Elsamitrucin + Bortezomib | 1.23 | 1.35 | 1.47 | Moderate Antagonism to | 1.61 | 1.80 | 2.02 | Antagonism |
| (Plate 2) | | | | | Antagonism | | | | |
| SPA-18 Daudi-e04 | Elsamitrucin + Camptothecin | 1.16 | 1.11 | 1.08 | Slight Antagonism to | 1.49 | 1.42 | 1.35 | Antagonism to Moderate |
| (Plate 3) | | | | | Nearly Additive | | | | Antagonism |
| SPA-21 Daudi-e06 | Elsamitrucin + Carmustine | 1.45 | 1.40 | 1.35 | Moderate Antagonism | 1.92 | 1.85 | 1.77 | Antagonism |
| (Plate 2) | | | | | | | | | |
| SPA-18 Daudi-e04 | Elsamitrucin + Cisplatin | 0.96 | 1.07 | 1.21 | Nearly Additive to | 1.18 | 1.35 | 1.55 | Slight Antagonism to |
| (Plate 5) | | | | | Moderate Antagonism | | | | Antagonism |
| SPA-18 Daudi-e04 | Elsamitrucin + Doxorubicin | 1.07 | 1.10 | 1.13 | Nearly Additive to Slight | 1.30 | 1.35 | 1.41 | Moderate Antagonism |
| (Plate 6) | | | | | Antagonism | | | | |
| SPA-19 Daudi-e05 | Elsamitrucin + Etoposide | 1.06 | 1.01 | 0.98 | Nearly Additive | 1.30 | 1.21 | 1.14 | Moderate Antagonism to |
| (Plate 1) | | | | | | | | | Slight Antagonism |
| SPA-19 Daudi-e05 | Elsamitrucin + Gemcitabine | 1.16 | 1.26 | 1.37 | Slight Antagonism to | 1.49 | 1.65 | 1.84 | Antagonism |
| (Plate 2) | | | | | Moderate Antagonism | | | | |
| SPA-19 Daudi-e05 | Elsamitrucin + Methotrexate | 1.62 | 1.57 | 1.53 | Antagonism | 2.27 | 2.17 | 2.09 | Antagonism |
| (Plate 3) | | | | | | | | | |
| SPA-19 Daudi-e05 | Elsamitrucin + Paclitaxel | 1.22 | 1.41 | 1.61 | Moderate Antagonism to | 1.60 | 1.90 | 2.26 | Antagonism |
| (Plate 4) | | | | | Antagonism | | | | |

TABLE 9 (Supplement)
Combination Index Values for Elsamitrucin and Selected Anticancer Agents
with the Daudi Human Lymphoma Cell Line

| | | Combination Index Value at | | | |
|---|---|---|---|---|---|
| | | Mutually Exclusive Model | | | |
| Experiment ID | Testing Agents | ED₅₀ | ED₇₅ | ED₉₀ | Summary |
| SPA-18 Daudi-e04 (Plate 1) | Elsamitrucin + 5-Fluorouracil | 1.26 | 1.13 | 1.10 | Moderate Antagonism to |
| | | | | | Slight Antagonism |
| SPA-18 Daudi-e04 (Plate 2) | Elsamitrucin + Bortezomib | 1.23 | 1.35 | 1.47 | Moderate Antagonism to |
| | | | | | Antagonism |
| SPA-18 Daudi-e04 (Plate 3) | Elsamitrucin + Camptothecin | 1.16 | 1.11 | 1.08 | Slight Antagonism to |
| | | | | | Nearly Additive |
| SPA-18 Daudi-e04 (Plate 4) | Elsamitrucin + Carmustine | 1.04 | 0.90 | 0.78 | Nearly Additive to |
| | | | | | Moderate Synergism |
| SPA-21 Daudi-e06 (Plate 1) | Elsamitrucin + Carmustine | 1.26 | 1.13 | 1.02 | Moderate Antagonism to |
| | | | | | Nearly Additive |
| SPA-21 Daudi-e06 (Plate 2) | Elsamitrucin + Carmustine | 1.45 | 1.40 | 1.35 | Moderate Antagonism |
| SPA-21 Daudi-e06 (Plate 3) | Elsamitrucin + Carmustine | 1.78 | 1.61 | 1.46 | Antagonism |
| SPA-18 Daudi-e04 (Plate 5) | Elsamitrucin + Cisplatin | 0.96 | 1.07 | 1.21 | Nearly Additive to |
| | | | | | Moderate Antagonism |
| SPA-18 Daudi-e04 (Plate 6) | Elsamitrucin + Doxorubicin | 1.07 | 1.10 | 1.13 | Nearly Additive to |
| | | | | | Slight Antagonism |
| SPA-19 Daudi-e05 (Plate 1) | Elsamitrucin + Etoposide | 1.06 | 1.01 | 0.98 | Nearly Additive |
| SPA-19 Daudi-e05 (Plate 2) | Elsamitrucin + Gemcitabine | 1.16 | 1.26 | 1.37 | Slight Antagonism to |
| | | | | | Moderate Antagonism |
| SPA-19 Daudi-e05 (Plate 3) | Elsamitrucin + Methotrexate | 4.62 | 1.57 | 1.53 | Antagonism |
| SPA-19 Daudi-e05 (Plate 4) | Elsamitrucin + Paclitaxel (1:0.125) | 1.22 | 1.41 | 1.61 | Moderate Antagonism to |
| | | | | | Antagonism |

| | Combination Index Value at | | | | |
|---|---|---|---|---|---|
| | Mutually Non-Exclusive Model | | | | |
| Experiment ID | ED₅₀ | ED₇₅ | ED₉₀ | Summary | Comment |
| SPA-18 Daudi-e04 (Plate 1) | 1.62 | 1.47 | 1.35 | Antagonism to | Presented in Table 9 |
| | | | | Moderate Antagonism | |
| SPA-18 Daudi-e04 (Plate 2) | 1.61 | 1.80 | 2.02 | Antagonism | Presented in Table 9 |
| SPA-18 Daudi-e04 (Plate 3) | 1.49 | 1.42 | 1.35 | Antagonism to | Presented in Table 9 |
| | | | | Moderate Antagonism | |
| SPA-18 Daudi-e04 (Plate 4) | 1.09 | 0.92 | 0.79 | Nearly Additive to | Carmustine solutions may have |
| | | | | Moderate Synergism | not been prepared appropriately |
| SPA-21 Daudi-e06 (Plate 1) | 1.57 | 1.37 | 1.20 | Antagonism to | |
| | | | | Moderate Antagonism | |
| SPA-21 Daudi-e06 (Plate 2) | 1.92 | 1.85 | 1.77 | Antagonism | Presented in Table 9 |
| SPA-21 Daudi-e06 (Plate 3) | 2.58 | 2.25 | 1.98 | Antagonism | Carmustine concentration |
| | | | | | range was slightly too high |
| SPA-18 Daudi-e04 (Plate 5) | 1.18 | 1.35 | 1.55 | Slight Antagonism to | Presented in Table 9 |
| | | | | Antagonism | |
| SPA-18 Daudi-e04 (Plate 6) | 1.30 | 1.35 | 1.41 | Moderate Antagonism | Presented in Table 9 |
| SPA-19 Daudi-e05 (Plate 1) | 1.30 | 1.21 | 1.14 | Moderate Antagonism to | Presented in Table 9 |
| | | | | Slight Antagonism | |
| SPA-19 Daudi-e05 (Plate 2) | 1.49 | 1.65 | 1.84 | Antagonism | Presented in Table 9 |
| SPA-19 Daudi-e05 (Plate 3) | 2.27 | 2.17 | 2.09 | Antagonism | Presented in Table 9 |
| SPA-19 Daudi-e05 (Plate 4) | 1.60 | 1.90 | 2.26 | Antagonism | Presented in Table 9 |

TABLE 10
Protocol Design for the HCT116-e256 Study

| | | Treatment Regimen 1 | | | | Treatment Regimen 2 | | | |
|---|---|---|---|---|---|---|---|---|---|
| Group | n | Agent | mg/kg | Route | Schedule | Agent | mg/kg | Route | Schedule |
| 1 | 10 | No Treatment | — | — | — | No Treatment | — | — | — |
| 2 | 10 | Elsamitrucin | 10 | ip | q4d x3 | — | — | — | — |
| 3 | 10 | Elsamitrucin | 5 | ip | q4d x3 | — | — | — | — |
| 4 | 10 | Paclitaxel | 15 | iv | qod x5 | — | — | — | — |
| 5 | 10 | Paclitaxel | 7.5 | iv | qod x5 | — | — | — | — |
| 6 | 10 | Cisplatin | 2.7 | ip | qd x5 | — | — | — | — |
| 7 | 10 | Cisplatin | 1.35 | ip | qd x5 | — | — | — | — |
| 8 | 10 | Elsamitrucin | 10 | ip | q4d x3 | Paclitaxel | 15 | iv | qod x5 |
| 9 | 10 | Elsamitrucin | 5 | ip | q4d x3 | Paclitaxel | 7.5 | iv | qod x5 |
| 10 | 10 | Elsamitrucin | 10 | ip | q4d x3 | Cisplatin | 2.7 | ip | qd x5 |
| 11 | 10 | Elsamitrucin | 5 | ip | q4d x3 | Cisplatin | 1.35 | ip | qd x5 |

TABLE 11
Treatment Response Summary for the HCT116-e256 Study

| | | Treatment Regimen 1 | | | | Treatment Regimen 2 | | | | Median |
|---|---|---|---|---|---|---|---|---|---|---|
| Group | n | Agent | mg/kg | Route | Schedule | Agent | mg/kg | Route | Schedule | TTE |
| 1 | 10 | No Treatment | — | — | — | No Treatment | — | — | — | 25.1 |
| 2 | 10 | Elsamitrucin | 10 | ip | q4d x3 | — | — | — | — | 27.1 |
| 3 | 10 | Elsamitrucin | 5 | ip | q4d x3 | — | — | — | — | 26.5 |
| 4 | 10 | Paclitaxel | 15 | iv | qod x5 | — | — | — | — | 39.8 |
| 5 | 10 | Paclitaxel | 7.5 | iv | qod x5 | — | — | — | — | 30.7 |
| 6 | 9 | Cisplatin | 2.7 | ip | qd x5 | — | — | — | — | 30.8 |
| 7 | 10 | Cisplatin | 1.35 | ip | qd x5 | — | — | — | — | 25.3 |
| 8 | 10 | Elsamitrucin | 10 | ip | q4d x3 | Paclitaxel | 15 | iv | qod x5 | 40.0 |
| 9 | 10 | Elsamitrucin | 5 | ip | q4d x3 | Paclitaxel | 7.5 | iv | qod x5 | 31.9 |
| 10 | 10 | Elsamitrucin | 10 | ip | q4d x3 | Cisplatin | 2.7 | ip | qd x5 | 26.3 |
| 11 | 10 | Elsamitrucin | 5 | ip | q4d x3 | Cisplatin | 1.35 | ip | qd x5 | 35.7 |

| | | | Statistical | MTV (n) | No. of | | | Mean BW | No. of | No. of |
|---|---|---|---|---|---|---|---|---|---|---|
| Group | T − C | % TGD | Significance | Day 59 | PR | CR | TFS | Nadir | TR | NTR |
| 1 | — | — | — | 0 (1) | 0 | 1 | 1 | — | 0 | 0 |
| 2 | 2.0 | 8% | ns | 88 (1) | 0 | 0 | 0 | −8.4% Day 7 | 0 | 0 |
| 3 | 1.4 | 6% | ns | — | 0 | 0 | 0 | −4.8% Day 7 | 0 | 0 |
| 4 | 14.7 | 59% | ns | — | 1 | 0 | 0 | −4.6% Day 7 | 0 | 0 |
| 5 | 5.6 | 22% | ns | — | 0 | 0 | 0 | — | 0 | 0 |
| 6 | 5.7 | 23% | ns | — | 0 | 0 | 0 | −6.9% Day 7 | 0 | 1 |
| 7 | 0.2 | 1% | ns | — | 0 | 0 | 0 | −0.9% Day 7 | 0 | 0 |
| 8 | 14.9 | 59% | ne | 0 (1) | 2 | 1 | 1 | −11.4% Day 10 | 2 | 0 |
| 9 | 6.8 | 27% | ns | — | 0 | 0 | 0 | −4% Day 7 | 0 | 0 |
| 10 | 1.2 | 5% | ns | — | 0 | 0 | 0 | −8.5% Day 7 | 0 | 0 |
| 11 | 10.6 | 42% | ns | — | 0 | 0 | 0 | — | 0 | 0 |

Study Endpoint = 2000 mm³, Days in Progress = 59
n = number of animals in a group not dead from accidental or unknown causes, or enthanized for sampling
MTV (n) = median tumor volume (mm³) for the number of animals on the day of TGD analysis (excludes animals attaining the tumor volume endpoint)
TTE = time to endpoint,
T − C = difference between median TTE (days) of treated versus control group,
% TGD = [(T − C)/C] × 100
Statistical Significance = Logrank test: ne = not evaluable, ns = not significant, * = P < 0.05, ** = P < 0.01, *** = P < 0.001, compared to Group 1
PR = partial regression,
CR = complete regression,
TFS = tumor free survivor
Mean BW Nadir = lowest group mean body weight, as % change from Day 1; — indicates no decrease in mean body weight was observed
TR = treatment-related death,
NTR = non-treatment-related death

What is claimed is:

1. A method of treating cancer comprising
administering elsamitrucin with one or more of 5-fluorouracil, bortezomib, camptothecin, carmustine, cisplatin, doxorubicin, etoposide, gemcitabine, methotrexate, and paclitaxel.

2. A method according to claim 1 wherein said elsamitrucin is administered with paclitaxel.

3. A method according to claim 1 wherein said elsamitrucin is administered with cisplatin.

4. A method according to claim 1 wherein said elsamitrucin comprises a salt form.

5. A method according to claim 4 wherein said salt form is a tosylate salt form or a succinate salt form.

5. A method according to claim 1 wherein said administering occurs in a mammal.

6. A method according to claim 5 wherein said mammal is selected from the group consisting of a human, a dog, a cat, a hamster, a guinea pig, a ferret and a pig.
